(19) United States
(12) Patent Application Publication
(10) Pub. No.: US 2023/0240715 A1
(43) Pub. Date: Aug. 3, 2023

(54) DELIVERY SYSTEMS FOR INJECTED NEUROSTIMULATION DEVICES, AND ASSOCIATED SYSTEMS AND METHODS

(71) Applicant: Invicta Medical, Inc., Santa Clara, CA (US)

(72) Inventors: Paul Paspa, Los Gatos, CA (US); Richard W. O'Connor, Atherton, CA (US); Timothy A. Fayram, Gilroy, CA (US); Dennis Potts, Scotts Valley, CA (US)

(21) Appl. No.: 18/104,739

(22) Filed: Feb. 1, 2023

Related U.S. Application Data
(60) Provisional application No. 63/306,025, filed on Feb. 2, 2022.

(51) Int. Cl. A61B 17/34; A61N 1/375; A61N 1/36

(52) U.S. Cl. CPC A61B 17/3468; A61B 17/3423; A61N 1/3756; A61N 1/3611; A61B 17/3403; A61B 2017/3443; A61B 2017/3413; A61B 2017/00477

(57) ABSTRACT

The present technology is generally directed to delivery systems for injected neurostimulation devices, and associated systems and methods. In some embodiments, a delivery system includes a body having an at least partially hollow interior; an adjustable sheath defining a lumen, the adjustable sheath including (i) a first portion positioned within the interior of the body and (ii) a second portion extending beyond the interior; and an actuation mechanism operably coupled to the body and the adjustable sheath. The actuation mechanism can be operable to move the adjustable sheath to change a length of the second portion. A user can operate the delivery system to position an implantable device at or near a target location within a patient. In some instances, the user may use image-based navigation and/or stimulation-based navigation to identify the position of the delivery system and/or the implantable device relative to target location.

### CROSS-REFERENCE TO RELATED APPLICATION(S)

The present application claims priority to U.S. Provisional Patent Application No. 63/306,025, titled “DELIVERY SYSTEMS FOR INJECTED NEUROSTIMULATION DEVICES, AND ASSOCIATED SYSTEMS AND METHODS,” and filed on Feb. 2, 2022, the entirety of which is hereby incorporated by reference herein.

### TECHNICAL FIELD

The present technology is directed generally to delivery systems for injected neurostimulation devices, and associated systems and methods. Representative embodiments include techniques for placing implantable electrodes, which are wirelessly coupled to a remote power delivery device, to treat sleep apnea. The delivery systems can be used to position implantable electrodes at target locations based at least in part on image-based navigation and stimulation-response-based navigation.

### BACKGROUND

Obstructive sleep apnea (OSA) is a medical condition in which a patient's upper airway is occluded (partially or fully) during sleep, causing sleep arousal. Repeated occlusions of the upper airway may cause sleep fragmentation, which in turn may result in sleep deprivation, daytime tiredness, and/or malaise. More serious instances of OSA may increase the patient's risk for stroke, cardiac arrhythmias, high blood pressure, and/or other disorders.

OSA may be characterized by the tendency for soft tissues of the upper airway to collapse during sleep, thereby occluding the upper airway. OSA is typically caused by the collapse of the patient's soft palate, oropharynx, tongue, epiglottis, or combination thereof, into the upper airway, which in turn may obstruct normal breathing and/or cause sleep arousal.

Some treatments have been available for OSA including, for example, surgery, constant positive airway pressure (CPAP) machines, and electrically stimulating muscles or related nerves associated with the upper airway to move the tongue (or other upper airway tissue). Surgical techniques have included procedures to remove portions of a patient's tongue and/or soft palate, and other procedures that seek to prevent the tongue from collapsing into the back of the pharynx. These surgical techniques are very invasive. CPAP machines seek to maintain upper airway patency by applying positive air pressure at the patient's nose and mouth. However, these machines are uncomfortable, cumbersome, and may have low compliance rates.

Some electrical stimulation techniques seek to prevent the tongue from collapsing into the back of the pharynx by causing the tongue to protrude forward (e.g., in an anterior direction) and/or flatten during sleep. However, existing techniques for electrically stimulating the nerves of the patient's oral cavity suffer from being too invasive, and/or not sufficiently efficacious. Thus, there is a need for an improved minimally-invasive treatment for OSA and other sleep disorders.

### BRIEF DESCRIPTION OF THE DRAWINGS

Representative embodiments of the present technology are illustrated by way of example and are not intended to be limited by the Figures, in which like reference numerals generally refer to corresponding parts throughout.

FIG. 1 is a side sectional view depicting a patient's upper airway.

FIG. 2 is a side view of a patient's skull, illustrating representative signal delivery targets in accordance with embodiments of the present technology.

FIG. 3A is a view of a patient's skull, from below, illustrating the hypoglossal nerve and a representative electrode location in accordance with embodiments of the present technology.

FIGS. 3B and 3C illustrate an approach for implanting a signal delivery device in accordance with embodiments of the present technology.

FIGS. 4A and 4B are side and side cross-section views, respectively, of an implantable device delivery system configured in accordance with embodiments of the present technology.

FIG. 5A is a side view of a navigation assembly configured in accordance with embodiments of the present technology.

FIGS. 5B and 5C are side cross-section views of the navigation assembly of FIG. 5A inserted into the implantable device delivery system of FIG. 4B, each in accordance with embodiments of the present technology.

FIG. 6A is a side view of an implantable device delivery assembly configured in accordance with embodiments of the present technology.

FIG. 6B is an exploded perspective view of a handle and a shaft end portion of a representative implantable device delivery assembly of FIG. 6A, each configured in accordance with embodiments of the present technology.

FIG. 6C is a perspective view of the handle and the shaft end portion shown in FIG. 6B, and selected elements of the delivery system 400 of FIG. 4A, in accordance with embodiments of the present technology

FIG. 7 is a side view of another implantable device delivery assembly configured in accordance with embodiments of the present technology.

FIGS. 8A-8C are side cross-section views of the implantable device delivery assembly of FIG. 6A carried and released by the implantable device delivery system, in accordance with embodiments of the present technology.

FIG. 9 is a flow-diagram of a process for positioning an implantable device at a target location using dual-mode navigation, in accordance with embodiments of the present technology.

FIGS. 10A and 10B illustrate a representative ultrasound probe, and associated image, in accordance with embodiments of the present technology.

FIGS. 11A and 11B illustrate a representative ultrasound probe, and associated image, in accordance with embodiments of the present technology.

FIG. 12 is a schematic illustration of a representative ultrasound probe used for processes in accordance with embodiments of the present technology.

### DETAILED DESCRIPTION

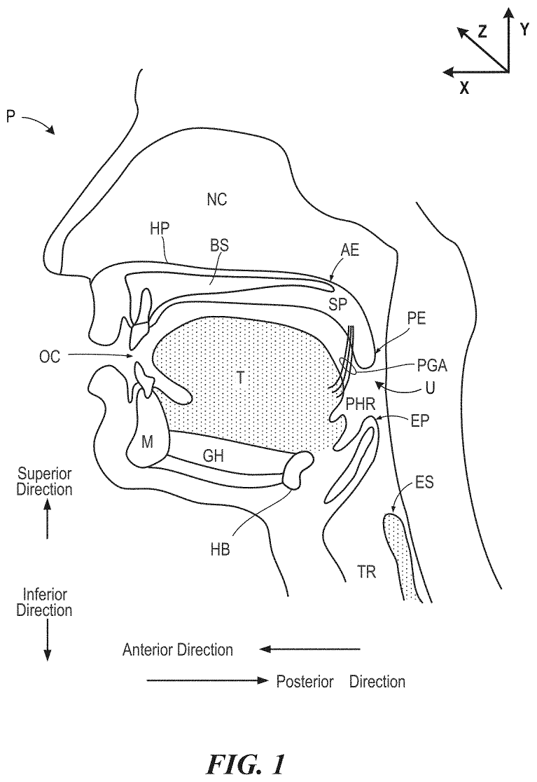

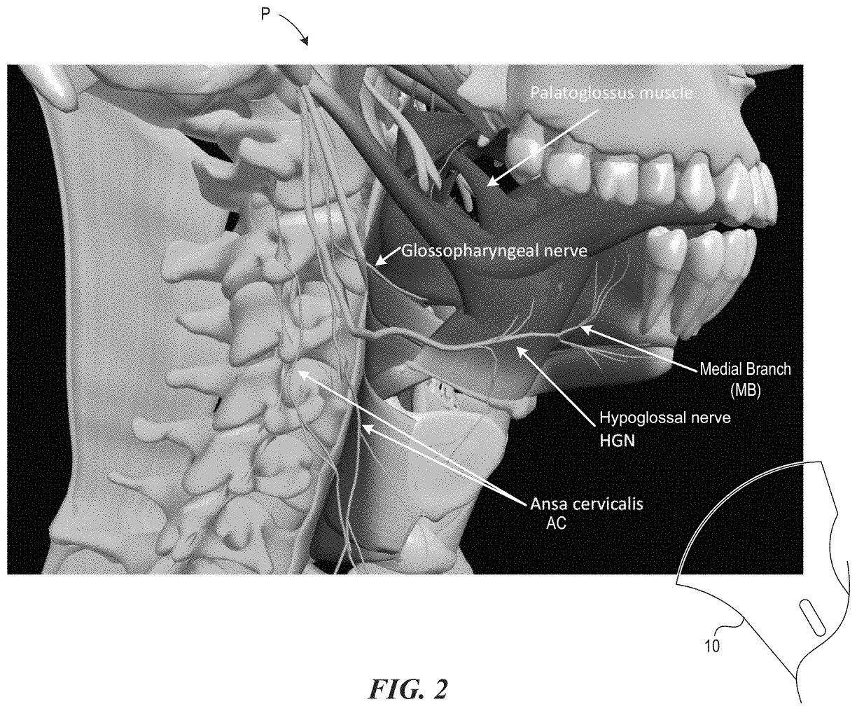

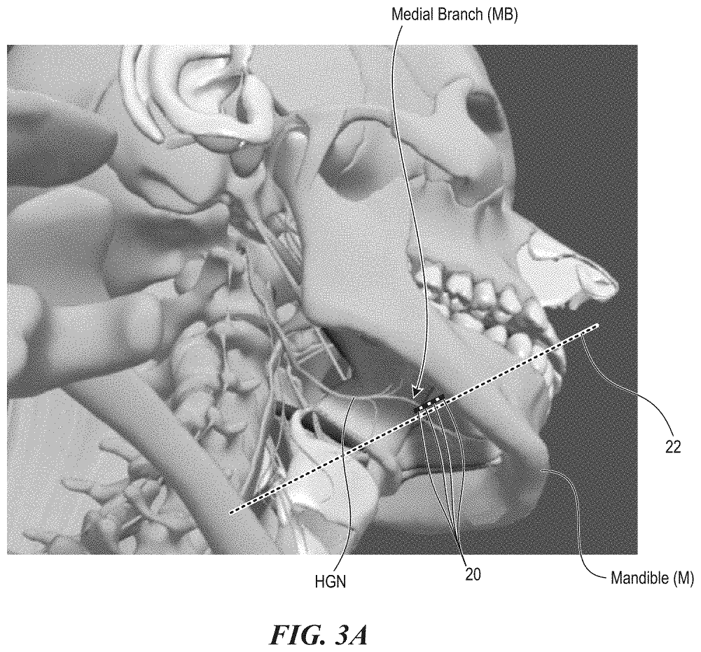

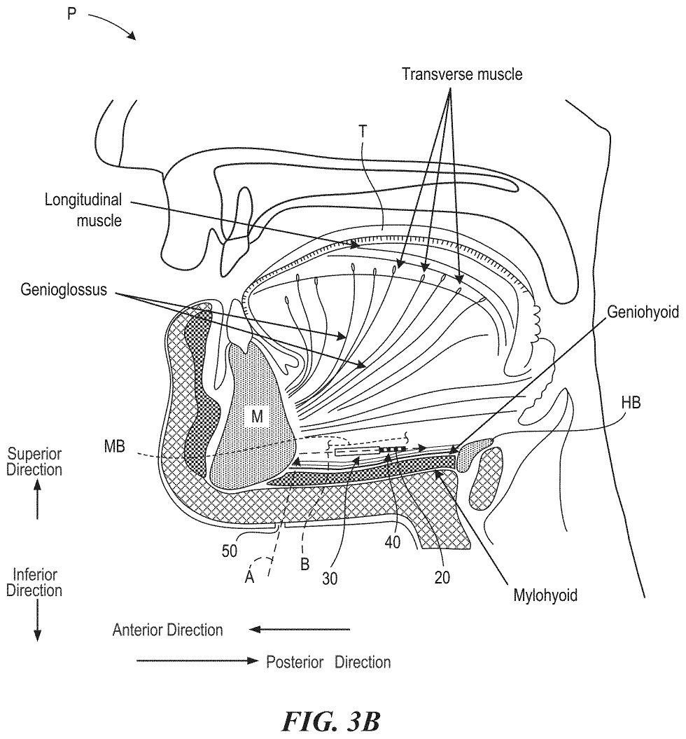

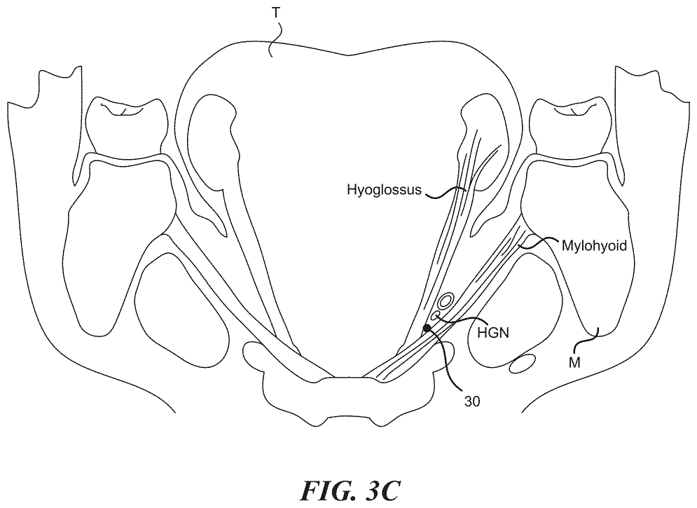

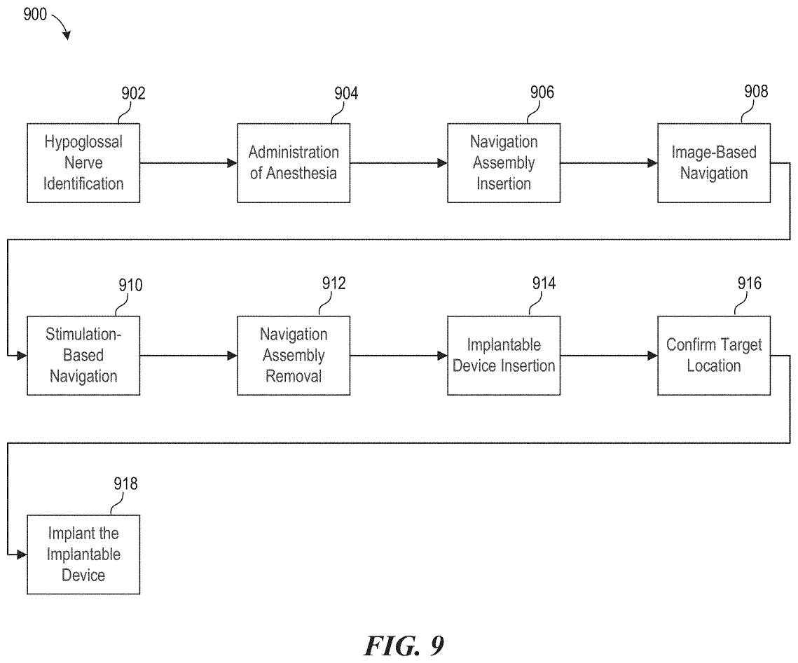

The present technology is discussed under the following headings for ease of readability:
Heading 1: “Introduction”
Heading 2: “Overall Patient Physiology” (with a focus on FIG. 1)
Heading 3: “Representative Stimulation Targets and Implant Techniques” (with a focus on FIGS. 2-3C)
Heading 4: “Overall System” (with a focus on FIGS. 4A-8C)
Heading 5: “Procedure” (with a focus on FIGS. 9-12)

While embodiments of the present technology are described under the selected headings indicated above, other embodiments of the technology can include elements discussed under multiple headings. Accordingly, the fact that an embodiment may be discussed under a particular heading does not necessarily limit that embodiment to only the elements discussed under that heading.

### 1. Introduction

Electrical stimulation for obstructive sleep apnea (OSA) typically includes delivering an electrical current that modulates nerves and/or muscles, e.g., to cause the tongue and/or other soft tissue to move. The electrical stimulation can accordingly remove an obstruction of the upper airway, or prevent the tongue or other soft tissue from collapsing or obstructing the airway. As used herein, the terms “modulate” and “stimulate” are used interchangeably to mean having an effect on, e.g., an effect on a nerve that in turn has an effect on one or more motor functions, e.g., a breathing-related motor function.

Representative methods and apparatuses for reducing the occurrence and/or severity of a breathing disorder, such as OSA, OSA with complete concentric collapse (“CCC”), central sleep apnea, and/or the like are disclosed herein. In accordance with representative embodiments, a minimally-invasive signal delivery device is implanted proximate to or adjacent to one or more tissues of the patient's upper airway, such as one or more nerves that innervate the patient's oral cavity, soft palate, oropharynx, and/or epiglottis. Representative nerves include the hypoglossal nerve, branches of the ansa cervicalis and/or the vagus nerves, which are located adjacent and/or around the oral cavity or in the neck. The signal delivery device can be implanted in the patient via a percutaneous injection. A non-implanted power source, e.g., including one or more mouthpiece portions, collar portions, chinstrap portions, pillow portions, mattress overlay portions, other suitable “wearables,” and/or one or more adhesive, skin-mounted devices, can wirelessly provide electrical power to the implanted signal delivery device. The signal delivery device emits accurately targeted electrical signals (e.g., pulses) that improve the patient's upper airway patency and/or improve the tone of the tissue of the intraoral cavity to treat sleep apnea. The electrical current delivered by the signal delivery device can stimulate at least a portion of a patient's hypoglossal nerve and/or other nerves and/or tissues (e.g., muscle tissue) associated with the upper airway. By moving the tongue forward and/or by preventing the tongue and/or soft tissue from collapsing onto the back of the patient's pharynx, and/or into the upper airway, the devices and associated methods disclosed herein can in turn improve the patient's sleep, e.g., by moving the potentially obstructing tissue in the upper airway/pharynx down. More specifically, applying the electrical signal to one or more portions of the hypoglossal nerve and/or directly to one or both of the patient's genioglossus muscles can cause the tongue to move forward/anteriorly (e.g., to produce a net positive protrusive response and/or increase flow through the patient's upper airway), and/or prevent, or at least partially prevent, the patient's tongue from collapsing onto the back of the patient's pharynx and/or into the upper airway. Applying the electrical signal to the ansa cervicalis can cause the thyroid and larynx move downward (inferiorly or caudally), a motion typically referred to as caudal traction.

Many embodiments of the technology described herein may take the form of computer- or machine- or controller-executable instructions, including routines executed by a programmable computer or controller. Those skilled in the relevant art will appreciate that the technology can be practiced on computer/controller systems other than those shown and described below. The technology can be embodied in a special-purpose computer, controller or data processor that is specifically programmed, configured or constructed to perform one or more of the computer-executable instructions described below. Accordingly, the terms “computer” and “controller” as generally used herein refer to any suitable data processor and can include Internet appliances and hand-held devices (including palm-top computers, wearable computers, tablets, cellular or mobile phones, multi-processor systems, processor-based or programmable consumer electronics, network computers, minicomputers and the like). Information handled by these computers can be presented at any suitable display medium, including a liquid crystal display (LCD).

The present technology can also be practiced in distributed environments, where tasks or modules are performed by remote processing devices that are linked through a communications network. In a distributed computing environment, program modules or subroutines may be located in local and remote memory storage devices. Aspects of the technology described below may be stored or distributed on any suitable computer-readable media, including one or more ASICs, (e.g., with addressable memory), as well as distributed electronically over networks. Data structures and transmissions of data particular to aspects of the technology are also encompassed within the scope of the embodiments of the technology.

### 2. Overall Patient Physiology

Representative embodiments described herein include signal delivery devices having electrodes that can be positioned to deliver one or more electrical currents to or toward one or more specific target locations, e.g., specific nerves and/or specific positions along a nerve. For example, the electrical current(s) can generate an electrical field that has an effect on the target nerve and/or other target tissue. FIG. 1 illustrates the general anatomy of the patient's oral cavity, and later Figures illustrate specific target locations. Such locations include locations along the patient's hypoglossal nerve, branches of the ansa cervicalis, and/or vagus nerves, as these nerves innervate muscles of the patient's airway (e.g., palatal, oropharyngeal, laryngeal muscles) besides the tongue. The target location can be identified with respect to any of, or any combination of, intrinsic or extrinsic muscles, associated nerve branches, and/or other physiological features. Such a target location and/or position can also be distal from the salivary glands (e.g., medial to the sublingual salivary gland) and/or other structures to avoid causing pain and/or other undesired effects.

FIG. 1 illustrates a patient P relative to a coordinate system in which the x-axis denotes the anterior-posterior directions, the y-axis denotes the superior-inferior directions, and the z-axis denotes the medial-lateral directions. The patient P has a hard palate HP which overlies the tongue T and forms the roof of the oral cavity OC (e.g., the mouth). The hard palate HP includes bone support BS, and thus does not typically deform during breathing. The soft palate SP, which is made of soft tissue such as membranes, fibrous material, fatty tissue, and muscle tissue, extends rearward (e.g., in a posterior direction) from the hard palate HP toward the back of the pharynx PHR. More specifically, an anterior end AE of the soft palate SP is anchored to a posterior end of the hard palate HP, and a posterior end PE of the soft palate SP is unattached. Because the soft palate SP does not contain bone or hard cartilage, the soft palate SP is flexible and may collapse onto the back of the pharynx PHR and/or flap back and forth (e.g., especially during sleep).

The pharynx PHR, which passes air from the oral cavity OC and the nasal cavity NC into the trachea TR, is the part of the throat situated inferior to (below) the nasal cavity NC, posterior to (behind) the oral cavity OC, and superior to (above) the esophagus ES. The pharynx PHR is separated from the oral cavity OC by the palatoglossal arch PGA, which runs downward on either side to the base of the tongue T. Although not shown for simplicity, the pharynx PHR includes the nasopharynx, the oropharynx, and the laryngopharynx. The nasopharynx lies between an upper surface of the soft palate SP and the wall of the throat (i.e., superior to the oral cavity OC). The oropharynx lies behind the oral cavity OC, and extends from the uvula U to the level of the hyoid bone HB. The oropharynx opens anteriorly into the oral cavity OC. The lateral wall of the oropharynx includes the palatine tonsil, and lies between the palatoglossal arch PGA and the palatopharyngeal arch. The anterior wall of the oropharynx includes the base of the tongue T and the epiglottic vallecula. The superior wall of the oropharynx includes the inferior surface of the soft palate SP and the uvula U. Because both food and air pass through the pharynx PHR, a flap of connective tissue called the epiglottis EP closes over the glottis (not shown for simplicity) when food is swallowed to prevent aspiration. The laryngopharynx is the part of the throat that connects to the esophagus ES, and lies inferior to the epiglottis EP. Below the tongue T is the lower jaw or mandible M, and the geniohyoid muscle GH, which is one of the muscles that controls the movement of the tongue T. The genioglossus muscle, which also controls tongue movement, and is a particular target of the presently disclosed therapy, is discussed later with reference to FIG. 3B.

### 3. Representative Stimulation Targets and Implant Techniques

FIG. 2 is a partially schematic, partially cut-away sagittal view of the neck and lower head region of the patient P. FIG. 2 illustrates representative neural structures of this region, including the hypoglossal nerve HGN (and its medial branch MB) and the ansa cervicalis AC. FIG. 2 also illustrates a representative ultrasound probe 10, used to aid in the process of positioning electrodes, which direct therapy signals to the target nerves.

FIG. 3A is a partially schematic, isometric illustration of the patient's skull, looking upwardly toward the mandible M. FIG. 3A also illustrates the hypoglossal nerve HGN which innervates the muscles controlling the patient's tongue T (FIG. 1). In representative embodiments, an electrode array comprising one or more electrodes 20 is positioned along the hypoglossal nerve HGN, in particular, at the medial branch MB of the HGN, in an electrode plane 22 defined by the medial branch MB. The medial branch MB innervates oral cavity muscles such as the genioglossus and geniohyoid muscles, which tend to pull the tongue forward (anteriorly), thus reducing the tendency for the soft tissue of the palate to prolapse into the patient's airway. By precisely positioning the electrode(s) 20 within this plane 22, and adjacent to the hypoglossal nerve HGN, it is expected that systems in accordance with embodiments of the present technology can more effectively control the patient's airway patency, without causing discomfort, and/or other undesirable effects, and/or in a manner that reduces the amount of power required to produce effective therapy signals. In some embodiments at least one of the electrodes 20 can be positioned to align with the medial branch MB. In other embodiments, the electrodes 20 can be centered relative to the medial branch MB. As discussed elsewhere herein, other representative target nerves include the ansa cervicalis and vagal nerves. Still further representative targets include cranial nerves (e.g., the glossopharyngeal nerve), and the palatoglossus muscle, which are shown in FIG. 2, and the genioglossus muscle, shown in FIG. 3B.

FIG. 3B is an enlarged view of the patient's lower jaw, illustrating the longitudinal and transverse muscles of the tongue T, as well as the genioglossus, geniohyoid, and mylohyoid muscles. An implantable device 30 can be inserted into the patient P. The implantable device 30 includes a signal delivery device 40, which in turn includes the one or more electrodes 20 that provide electrical stimulation to the target neural population. The electrodes 20 can be carried by a lead, or the implantable device 30 can be leadless. The implantable device 30 and/or the signal delivery device 40 can be configured to receive power wirelessly, such as from a power source positioned external to the patient P. Additional details regarding the implantable device 30 can be found in U.S. patent application Ser. No. 17/518,414, filed Nov. 3, 2021; and U.S. patent application Ser. No. 17/749,025, filed May 19, 2022; the entireties of which are incorporated by reference herein.

The implantable device 30 is shown after it has been inserted into the patient P, so as to be positioned inferior to the genioglossus at the intersection of the genioglossus and the geniohyoid muscles. The signal delivery device 40 is also adjacent to the medial branch MB, which is shown schematically in dashed lines. In FIG. 3B, the implantable device 30 was introduced into the patient (by e.g., a clinician or practitioner) by forming an opening 50 in the patient's skin and adjacent tissue. Although the opening 50 is shown as being positioned be anteriorly relative to medial branch MB in FIG. 3B, in other embodiments the opening 50 can positioned posteriorly relative to the medial branch MB, medial-laterally relative to the medial branch MB, and/or can have any other suitable position.

The practitioner can insert the implantable device 30 through the opening 50 along a first path portion or trajectory, in a superior or generally superior direction, as indicated by the arrow A. When moving along the first path portion, the implantable device 30 can be perpendicular or generally perpendicular to the medial branch MB and at least partially pass through or penetrate one or more of the patient's tissues. In the illustrated embodiment, for example, the first path portion passes at least partially through the patient's Mylohyoid muscle. As described in greater detail below regarding FIG. 9, the penetration of one or more of the patient's tissues can be visualized using the ultrasound probe 10 and can be used to determine the position of the implantable device 30 during insertion.

Once the implantable device 30 has traveled a predetermined or otherwise suitable distance along the first path portion, the implantable device 30 can be transitioned or reoriented from the first path portion to a second path portion, indicated by arrow B, such that the implantable device 30 is parallel or generally parallel to the medial branch MB. In the illustrated embodiment, for example, transitioning/reorienting the implantable device 30 from the first path portion to the second path portion includes rotating or pivoting the implantable device in the posterior direction. In other embodiments, transitioning/reorienting the implantable device 30 from the first path portion to the second path portion can include rotating, pivoting, or otherwise moving the implantable device in any other suitable direction. In some embodiments, the practitioner reorients the implantable device 30 by manually rotating the implantable device 30. In other embodiments, the practitioner can insert the implantable device 30 using a curved tool that by virtue of its shape, reorients the implantable device 30. In at least some embodiments, however, the insertion path can have a single portion that is unidirectional, or at least generally unidirectional.

The practitioner can move the implantable device 30, e.g., along the second path portion or another suitable path portion, until the signal delivery device 40 is at a precise location relative to the medial branch MB (shown schematically in dotted lines in FIG. 3B). The precise location can be identified by applying an electrical signal to the signal delivery device and observing the patient's response (e.g., motor response, nerve twitching, and the like). The practitioner can use ultrasound and/or another suitable visualization technique, in addition to or in lieu of inducing a patient response. Any of these techniques can be performed iteratively until the electrodes 20 carried by the signal delivery device 40 are properly positioned. For example, in a representative process, the practitioner uses ultrasound to position the signal delivery device 40 close to the target location, and then iteratively applies the electrical signal while incrementally moving the signal delivery device and observing the patient's response until the target location is more precisely identified.

Although in FIG. 3B the implantable device 30 is shown as being inserted into the patient P in an anterior-to-posterior direction, in other embodiments the implantable device 30 can be inserted into the patient P in a posterior-to-anterior direction, an inferior-to-superior direction, a superior-to-inferior direction, a combination thereof, and/or another suitable direction. Additionally or alternatively, although in FIG. 3B the opening 50 is formed through an underside of the patent's jaw (e.g., inferior of the patient's mandible M), in other embodiments the opening 50 can be formed intraorally, such as below the patient's tongue T and/or at another suitable location.

In some embodiments, multiple openings can be made in the patient's skin and adjacent tissue and used to position the implantable device, such as described with reference to FIG. 4B in U.S. patent application Ser. No. 17/749,025, incorporated by reference herein. In such embodiments, for example, a bidirectional cannula can insert the implantable device percutaneously and then exit percutaneously at a distance from the entry on a sector arc of a specified radius. The implantable device can then be attached at the exit wound and, when the bidirectional cannula is pulled back to release the implantable device, the implantable device can be pulled into position by a tether, e.g., rather than “pushing” the implantable device into position as described above. Small stimulation electrodes could be located at the midsection of the tether.

FIG. 3C is a coronal view taken through the patient's oral cavity, illustrating the implantable device 30 in a representative position. The device 30 is seen in cross-section as it extends into and out of the plane of FIG. 3C. In this position, the device 30 is just lateral from the hyoglossus and just medial from the mylohyoid at or near the point at which the planes of these two muscles cross. The device 30 is positioned just inferior to the HGN, which also extends into and out of the plane of FIG. 3C.

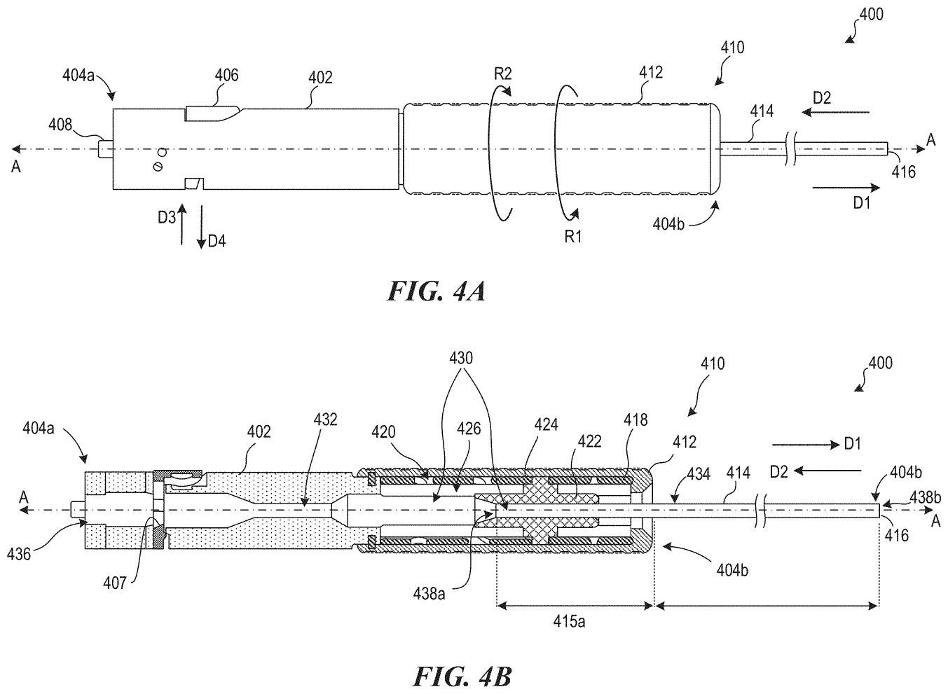

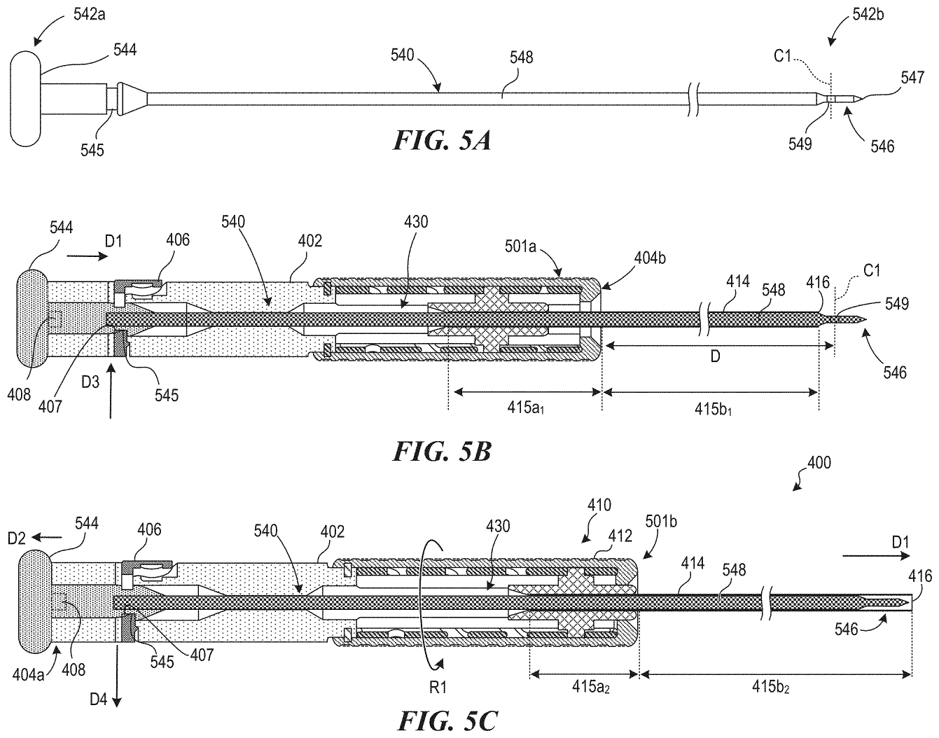

An advantage of the foregoing approach is that the practitioner can move the signal delivery device 40 to find a precise target location, via a single opening in the patient. Additionally, the signal delivery device is introduced into the patient percutaneously, which can improve patient outcomes, for example, by reducing the likelihood for an infection to develop. Although FIGS. 3B and 3C are described with reference to inserting and positioning the implantable device 30, in some embodiments the practitioner can first position an electrode of a navigation assembly relative to the medial branch MB, and then replace the electrode with the implantable device 30, as discussed later with reference to FIGS. 5A-5C and 9.

Any of the techniques described herein for implanting the signal delivery device 40 can include one or more additional operations. For example, the practitioner can compress or otherwise manipulate (e.g., with his/her fingers) the submandibular or intraoral tissue to facilitate positioning the signal delivery device. These methods can allow the practitioner to manipulate the insertion trajectories toward a desired endpoint. The additional force can be in the form of manual pressure applied intra- or extraorally, and/or a vacuum force that is targeted to move tissue as a way of improving the precision with which the signal delivery device is implanted. Pressure and/or suction can also be used to avoid internal structures, such as glands.

The foregoing discussion with reference to FIGS. 2-3C focused on electrodes positioned to deliver signals to the medial branch MB of the hypoglossal nerve. As discussed previously, it is expected to be advantageous to apply electrical signals to other tissues, such as the ansa cervicalis, e.g., individual ones of the branches of the ansa cervicalis that innervate the omohyoid, the sternothyroid, and the sternohyoid muscles, the patient's genioglossus muscle(s), other tissues described herein, and/or other suitable target locations, in addition to or in lieu of applying signals to the medial branch. Accordingly, the approach discussed above with reference to FIGS. 3A-3C can also be used to position the signal delivery device 40 proximate to the ansa cervicalis.

### 4. Overall System

FIGS. 4A and 4B are side and side cross-section views, respectively, of an implantable device delivery system 400 (“the delivery system 400”) configured in accordance with embodiments of the present technology. The delivery system 400 can be used to (i) identify/locate a target implant location within a patient, and also (ii) implant an implantable device, such as the implantable device 30 of FIGS. 3B and 3C, at the target location. In at least some embodiments, for example, the delivery system 400 can be used to identify a target location proximate the medial branch MB (FIGS. 3A-3C) of a patient's hypoglossal nerve, and then position the implantable device 30 (FIGS. 3B and 3C) proximate the medial branch MB, e.g., to address the patient's sleep apnea. As described in greater detail below, the delivery system 400 is expected to implant the implantable device at the target location with improved accuracy, improved repeatability, and/or reduced implantable device migration compared to other implantable device delivery systems. In at least some embodiments, for example, once a target location is identified based at least partially on, e.g., a patient's motor response, the delivery system 400 can then position the implantable device 30 at or near the target location such that the implantable device 30 can elicit the same or a generally similar motor response in the patient.

Referring to FIG. 4A, the delivery system 400 includes a body or housing 402 having a first (e.g., proximal) end portion 404a and a second (e.g., distal) end portion 404b opposite from the first end portion 404a. The first end portion 404a and the second end portion 404b can at least partially define a longitudinal axis A of the delivery system 400. The delivery system 400 further includes an adjustable delivery sheath or cannula 414 (“the adjustable cannula 414”). The adjustable cannula 414 can have a distal end terminus 416 that is spaced apart from the distal end portion 404b of the housing 402. The distal end terminus 416 can be atraumatic and/or sloped or tapered inwardly toward the longitudinal axis A. In the illustrated embodiment, the adjustable cannula 414 is straight and generally parallel (e.g., colinear) with longitudinal axis A. In other embodiments, the adjustable cannula 414 can be curved relative to the longitudinal axis A. For example, in at least some embodiments, the adjustable cannula 414 can include a fixed radius of curvature. The fixed radius of curvature can be relatively slight (e.g., a relatively large radius) and configured to assist the implanter with orienting the implantable device relative to the implant location during insertion. For example, the fixed radius of curvature can be selected so as to automatically transition the implantable device from the first path portion (arrow A in FIG. 3B) to the second path portion (arrow B in FIG. 3B), as described previously herein. Additionally, or alternatively, the adjustable cannula 414 can be at least partially segmented and configured such that the radius/curvature of the cannula 414 can be adjustable at least during an implantation procedure, e.g., in response to an axial force applied by a practitioner. For example, a practitioner can apply a force to (e.g., push) the segmented cannula to decrease the segmented cannula's radius of curvature and cause the segmented cannula to bend or curve. In some embodiments, the practitioner can apply a side force to steer the segmented cannula in a desired direction. In these and other embodiments, the adjustable cannula can have a spiral shape/configuration such that a practitioner can twist or “screw” the delivery system 400 while maneuvering the implantable device 30 into position proximate the medial branch. In these and other embodiments, the adjustable cannula 414 can be formed from polyether ether ketone (PEEK), or any other suitable material.

The adjustable cannula 414 can be operably coupled to an actuation mechanism 410. The actuation mechanism 410 can include an actuator 412 that, when actuated, moves or adjusts the adjustable cannula 414 relative to the housing 402. In the illustrated embodiment, for example, the actuator 412 is rotatably coupled to the housing 402. In operation, rotating the actuator 412 in a first rotary direction R1 can move the distal end terminus 416 in a first axial (e.g., distal) direction D1 away from the housing 402. Additionally, rotating the actuator 412 in a second rotary direction R2 opposite the first rotary direction R1 can move the distal end terminus 416 in a second axial (e.g., proximal) direction D2 opposite the first axial direction D1 and toward the housing 402. Accordingly, the adjustable cannula 414 can be bidirectional, e.g., configured to move in at least two directions relative to the housing 402. The first and second axial directions D1, D2 can each be parallel to the longitudinal axis A. In other embodiments, the actuator 412 can be operably coupled to the housing 402 and/or the adjustable cannula 414 via other mechanisms, such as a mechanical mechanism (e.g., a rack and pinion, a worm gear, a drive belt, a linear motor, etc.) a hydraulic mechanism, a pneumatic mechanism, an electrical mechanism, and/or another suitable mechanism. Additionally, or alternatively, in some embodiments the actuator 412 can be moved linearly, e.g., in a direction parallel, or at least generally parallel, to the longitudinal axis A, to move/adjust the adjustable cannula 414 relative to the housing 402. In such embodiments, the actuator 412 can be configured so that moving the actuator 412 in the first axial direction D1 moves the adjustable cannula 414 in one of the first axial direction D1 or the second axial direction D2, and moving the actuator 412 in the second axial direction D2 moves the adjustable cannula 414 in the other of the first axial direction D1 or the second axial direction D2.

Referring to FIG. 4A, the delivery system 400 can further include a latch component 406 and/or a release component 408. The latch component 406 can include a retaining surface 407 (FIG. 4B) configured to releasably couple the delivery system 400 to one or more tools or assemblies received within the delivery system 400, as described in greater detail below with reference to FIGS. 5A-8C. In the illustrated embodiment, the latch component 406 includes one or more springs or coupling elements configured to bias the latch component 406 radially relative to the longitudinal axis A in a third direction D3 to couple to a tool (described later with reference to FIGS. 5A-5C), and the latch component 406 can be moved or “pressed” radially relative to the longitudinal axis A in a fourth direction D4 opposite the third direction D3 to decouple the tool. The release component 408 can include one or more springs or other biasing elements configured to bias the release component 408 in the second axial direction D2. The release component 408 can be moved or “pressed” in the first axial direction D1 when a tool is received within the delivery system 400. The release component 408 can be held in this “pressed” state by the latch component 406 and any of the inserted tools 540, 650, or 750, which are described in greater detail below regarding FIGS. 5A-7, respectively. When the latch component 406 is actuated to decouple a tool, the release component 408 pushes the tool in the second axial direction D2 so the tool (e.g., a retaining surface 545 of the tool 540, see FIG. 5A) clears the retaining surface 407 to decouple the tool from the delivery system 400 and allow a user to more easily remove the tool from the delivery system 400.

Referring to FIG. 4B, the actuation mechanism 410 further includes a drive element 418 and a corresponding driven element 422. The driven element 422 can be coupled to the adjustable cannula 414 and operably coupled to the drive element 418. The drive element 418 can be operably coupled to the actuator 412. In the illustrated embodiment, the drive element 418 includes a collar element that defines one or more helical or spiral channels 420 around the longitudinal axis A that extend in a direction generally parallel to the longitudinal axis A, and the driven element 422 is a sliding element slidably disposed within the housing 402 and including one or more tabs 424. Individual ones of the tabs 424 can extend through a linear slot 426 within the housing 402 to be slidably received by a corresponding one of the spiral channels 420. The linear slot 426 can at least partially prevent the driven element 422 from rotating relative to the housing 402, such that rotational movement of the spiral channels 420 can drive linear movement of the tabs 424 when the drive element 418 is rotated. Accordingly, rotating the drive element 418 can drive linear movement of the driven element 422 in the first and second directions D1, D2 relative to the housing 402. Because the driven element 422 is coupled to the adjustable cannula 414, moving the driven element 422 moves the distal end terminus 416 of the adjustable cannula 414 relative to the housing 402 as described previously. In other embodiments, the actuator 412 can be a linear actuator slidably coupled to the housing 402 (via, e.g., one or more rack and/or ring-and-pinion sets), operably coupled to the adjustable cannula 414, and configured to (i) move in the first direction D1 to cause the distal end terminus 416 to move in the first direction D1 and (ii) move in the second direction D2 to cause the distal end terminus 416 to move in the second direction D2. In other embodiments, the actuator 412 can have other suitable configurations.

In some embodiments, at least a portion of the adjustable cannula 414 can be positioned within the housing 402. In the illustrated embodiment, for example, an inner portion or length 415a of the adjustable cannula 414 is positioned within the housing 402, and the adjustable cannula 414 further includes an outer portion or length 415b positioned outside of and/or extending from the housing 402 (e.g., the second end portion 404b of the housing 402). Accordingly, actuating the actuator 412, such as described above, can cause corresponding changes to the respective inner and outer lengths 415a, 415b of the adjustable cannula 414. In the illustrated embodiment, for example, moving the distal end terminus 416 in the first axial direction D1 increases the outer length 415b and decreases the inner length 415a by the same amount. Similarly, moving the distal end terminus 416 in the second axial direction D2 decreases the outer length 415b and correspondingly increases the inner length 415a by the same amount. In such embodiments, moving the distal end terminus 416 in the first axial direction D1 can also be referred to as advancing the adjustable cannula 414, and moving the distal end terminus 416 in the second axial direction D2 can also be referred to as retracting the adjustable cannula 414.

The delivery system 400 can further include an overall lumen 430. The overall lumen 430 can be defined by the housing 402 and the adjustable cannula 414, and can extend fully through the housing 402 and the adjustable cannula 414, e.g., between the first end portion 404a and the distal end terminus 416. In the illustrated embodiment, the overall lumen 430 includes a first or housing lumen 432 at least partially defined by the housing 402 and a second or cannula lumen 434 at least partially defined by the adjustable cannula 414. The housing lumen 432 can extend between an opening 436 in the first end portion 404a of the housing 402 and a first or proximal opening 438a of the adjustable cannula 414. In the illustrated embodiment, the first opening 438a is positioned within the driven element 422. In other embodiments, the first opening 438a can have any other suitable position within the delivery system 400. The cannula lumen 434 can extend between the first opening 438a and a second or distal opening 438b of the adjustable cannula 414 proximate the distal end terminus 416. The housing lumen 432 and the cannula lumen 434 can be aligned such that at least a portion of an elongated object or tool can be inserted into the housing 402 via the opening 436 and pass at least partially through the housing lumen 432 and the cannula lumen 434.

FIG. 5A is a side view of an obturator or navigation assembly 540 configured in accordance with embodiments of the present technology. The navigation assembly 540 is a representative tool that is configured to be inserted at least partially within an implantable device delivery system, such as the delivery system 400 of FIGS. 4A and 4B. The navigation assembly 540 can have a first (e.g., proximal) end portion 542a including a handle 544, a second (e.g., distal) end portion 542b including a stimulating component 546, and an elongate body 548 extending therebetween. The stimulating component 546 can include a distal end terminus or tip 547 and at least one electrode 549 (e.g., a navigation electrode) spaced proximally apart from the distal tip 547. The navigation electrode 549 can have a center C1, shown using a dashed line in FIG. 5A. In some embodiments, the navigation assembly 540 can include a recess or notch 545 positioned proximally relative to the handle 544. The notch 545 can be configured to releasably receive at least a portion of the latch component 406 (FIG. 4A) e.g., to releasably couple the navigation assembly 540 to the delivery system 400 (FIGS. 4A and 4B).

In operation, such as during any of the implantable device delivery procedures described herein, the navigation assembly 540 can be used to navigate to possible implantation sites (using, e.g., ultrasound) and evaluate these implantation sites (using, e.g., electrical stimulation). For example, the stimulating component 546 can be visualized using ultrasound while the practitioner navigates toward the medial branch MB, and the navigation electrode 549 can be coupled to an electrical power source (e.g., a peripheral nerve stimulator or signal delivery generator) and activated to deliver one or more stimulation bursts to the patient's surrounding tissue(s) to, for example, induce a motor response in the patient. In some embodiments, the distal tip 547 can be sharp and used to initially penetrate the patient's skin. In other embodiments, the distal tip 547 can be blunt or atraumatic and a trocar or other device can be used to initially penetrate the patient's skin.

FIGS. 5B and 5C are side cross-section views of the navigation assembly 540 of FIG. 5A and the delivery system 400 of FIG. 4A in accordance with embodiments of the present technology. Specifically, FIG. 5B shows the delivery system 400 in a first configuration 501a, and FIG. 5C shows the delivery system 400 in a second configuration 501b. Referring to FIGS. 5B and 5C together, the navigation assembly 540 can be sized or otherwise dimensioned to be at least partially insertable into the overall lumen 430 of the delivery system 400. In the illustrated embodiment, for example, the stimulating component 546 of the navigation assembly 540 has been inserted into the opening 436 (FIG. 4B) of the housing 402 and moved through the overall lumen 430 such that the stimulating component 546 is proximate the distal end terminus 416. During insertion, the notch 545 can operably engage the latch component 406 to secure the navigation assembly 540 relative to the delivery system 400.

Referring to FIG. 5B, when the delivery system 400 is in the first configuration 501a, the stimulating component 546 can extend through the adjustable cannula 414 beyond the distal end terminus 416, such that the stimulating component 546 can stimulate patient tissues, e.g., to induce a motor response. The center C1 of the navigation electrode 549 (FIG. 5A) can be a distance D from the second end portion 404b of the housing 402. When the navigation assembly 540 is inserted into the delivery system 400, the latch component 406 can be biased in the third direction D3 such that the retaining surface 407 is positioned at least partially within the notch 545, e.g., to couple the navigation assembly 540 to the delivery system 400. Additionally, or alternatively, the handle 544 can contact the release component 408 and move or depress the release component 408 in the first direction D1, as described previously regarding FIG. 4A.

Referring to FIG. 5C, the actuation mechanism 410 can be actuated to transition the delivery system 400 from the first configuration 501a to the second configuration 501b. In the second configuration 501b, the stimulating component 546 can be positioned at least partially or fully within the adjustable cannula 414. In the illustrated embodiment, transitioning the delivery system 400 to the second configuration 501b includes rotating the actuator 412 in the first direction R1 to move the distal end terminus 416 of the cannula 414 in the first direction D1, as described previously. Transitioning the delivery system 400 to the second configuration 501b can also change the inner and outer lengths 415a, 415b of the adjustable cannula 414. For example, transitioning the delivery system 400 from the first configuration 501a to the second configuration 501b can increase the outer length 415b from a first outer length 415b₁ (FIG. 5B) to a second outer length 415b₂. Additionally, transitioning the delivery system 400 from the first configuration 501a to the second configuration 501b can decrease the inner length 415a from the first inner length 415a₁ (FIG. 5B) to the second inner length 415a₂. The inner and outer lengths 415a, 415b can change by a same amount when the delivery system 400 transitions between the first and second configurations 501a, 501b. For example, the increase in the outer length 415b can be the same as the decrease in the inner length 415a.

In operation, with the navigation assembly 540 inserted into the delivery system 400, the delivery system 400 can be transitioned from the first configuration 501a to the second configuration 501b prior to removing the navigation assembly 540 from the delivery system 400. To prepare to remove the navigation assembly 540, the latch component 406 can be moved in the fourth direction D4 to separate the retaining surface 407 from the notch 545 and uncouple the navigation assembly 540 from the delivery system 400. In some embodiments, moving the latch component 406 in the fourth direction D4 allows the release component 408 (shown in dashed line in FIGS. 5B and 5C; best seen in FIG. 4A) to move in the second direction D2, as described above regarding FIG. 4A, such that the release component can move the navigation assembly 540 in the second direction D2 to create a gap (not shown in FIG. 5C) between the handle 544 and the first end portion 404a, e.g., to allow the user to more easily remove the navigation assembly 540 from the delivery system 400.

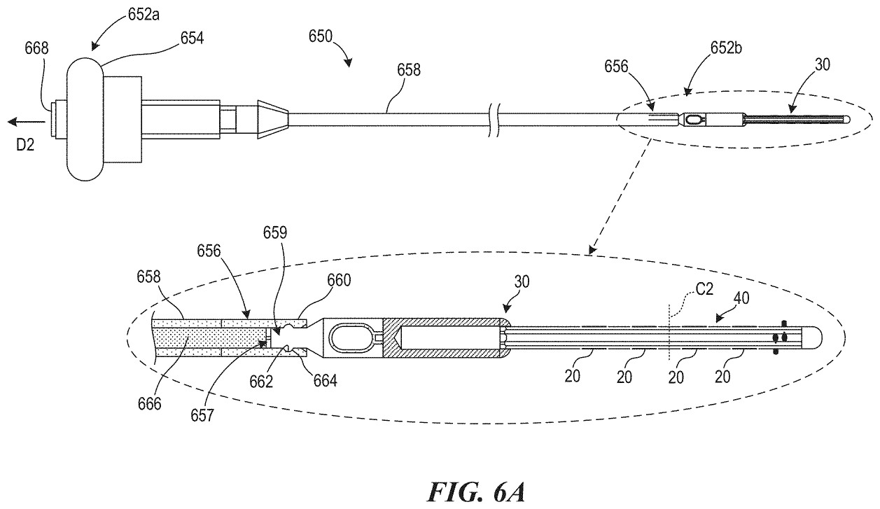

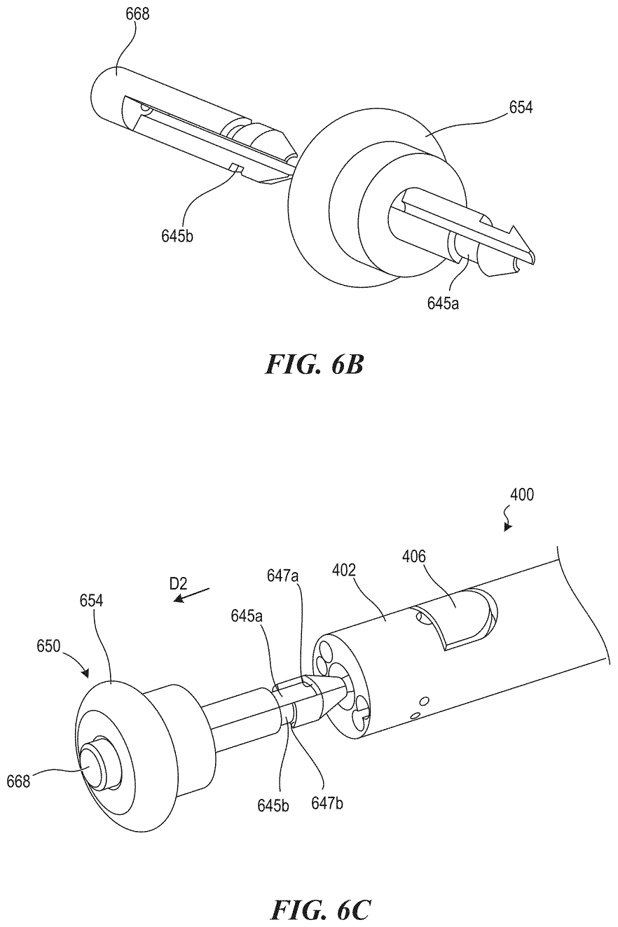

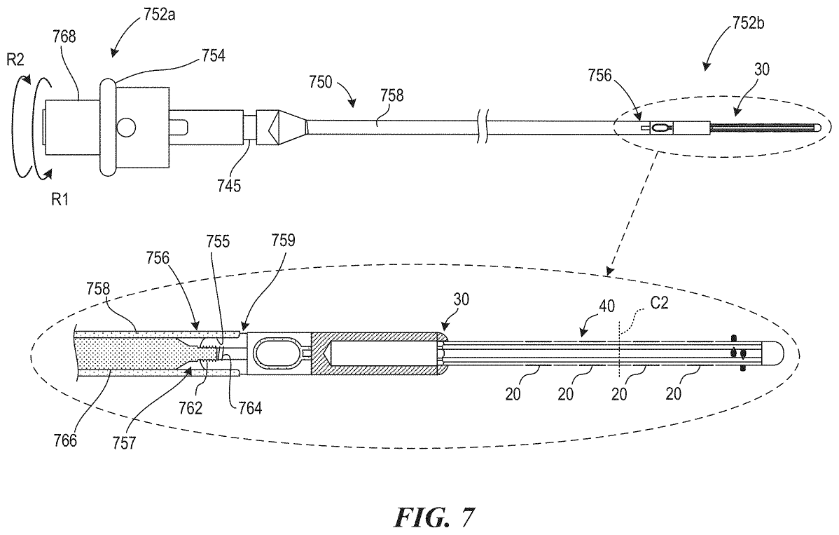

FIG. 6A is a side view of an implantable device insertion or delivery assembly 650 (“the implantable delivery assembly 650”) configured in accordance with embodiments of the present technology. FIG. 6A also includes an enlarged, cross-sectional view of the circled portion of the implantable delivery assembly 650 (shown in dashed lines). The implantable delivery assembly 650 can be insertable at least partially within an implantable device delivery system, such as the delivery system 400 shown in FIGS. 4A and 4B. The implantable delivery assembly 650 has a first (e.g., proximal) end portion 652a including a handle 654, a second (e.g., distal) end portion 652b including an implantable device coupling component 656 (“the coupling component 656”), and an elongate body 658 therebetween. The coupling component 656 can be configured to releasably connect to an implantable device, such as the implantable device 30 of FIG. 3B. Accordingly, the implantable delivery assembly 650 can be used to carry, move, or otherwise position the implantable device 30 at or near a target location within a patient, such as proximate a medial branch MB of a patient's hypoglossal nerve HGN (FIG. 3B). In FIG. 6A, at least a portion of the implantable device's internal components are omitted from the cross-sectional view for the purpose of illustrative clarity. It will be appreciated, however, that in practice the implantable device 30 can include one or more internal components, such as one or more of the components described with reference to FIGS. 6A and 6B in U.S. patent application Ser. No. 17/749,025, incorporated herein by reference.

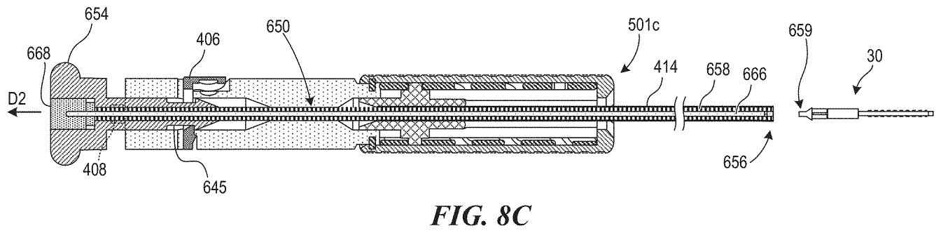

Additionally, the signal delivery device 30 can have a center C2, shown using a dashed line in FIG. 6A. As discussed above with reference to FIG. 3B, the signal delivery device 40 can carry one or more electrodes 20. The center of the electrode array C2 can be used to align the signal delivery device 30 with the target nerve during the implant procedure, as described further below with reference to FIGS. 8A-8C. Although in FIG. 6A the implantable device 30 is illustrated as including a lead carrying the electrodes 20, in other embodiments the lead can be omitted, e.g., the implantable device 30 can be leadless and can accordingly carry the electrodes 20 without a flexible lead.

The coupling component 656 can include a cavity or chamber 657 defined at least partially by one or more retaining or coupling elements 660 extending distally from the elongate body 658. The chamber 657 can be sized to receive at least a coupling portion 659 of the implantable device 30. In the illustrated embodiment, each of the coupling elements 660 includes a notch or recess 662 sized to receive a corresponding protrusion or tab 664 of the coupling portion 659 of the implantable device 30. Additionally, or alternatively, individual ones of the coupling elements 660 can include one or more tabs 664 and the implantable device 30 can include one or more corresponding recesses 662. In these and other embodiments, each of the recesses 662 can receive the corresponding tab 664 when the coupling portion 659 of the implantable device 30 is inserted into the chamber 657. The mechanical interaction (e.g., force, friction, interference fit, or the like) between the recesses 662 and the tabs 664 can releasably couple the implantable device 30 to the implantable delivery assembly 650. Additionally, or alternatively, individual ones of the coupling elements 660 can include an inwardly-biased leaf spring, or any other suitable biasing element, configured to “pinch” or otherwise apply a force to the coupling portion 659 when the coupling portion 659 is inserted in the chamber 657. The force applied to the coupling portion 659 by the coupling elements 660 can maintain the connection between the recesses 662 and the tabs 664 and/or at least partially prevent the implantable device 30 from moving relative to the implantable delivery assembly 650.

The coupling component 656 further includes a rod or shaft 666 slidably disposed within the elongate body 658 and extending at least partially between the first and second end portions 652a, 652b. The shaft 666 can be coupled to a shaft end portion 668. The shaft end portion 668 can be slidably disposed within the handle 654. The handle 654 can be coupled to the elongate body 658.

To release the implantable device 30 from the implantable delivery assembly 650, the handle 654 can be moved in the second direction D2 to cause a corresponding movement of the elongate body 658 and the coupling elements 660 in the second direction D2. The shaft 666 can be held stationary relative to the elongate body 658 as the elongate body 658/handle 654 are moved in the second direction D2. For example, in the illustrated embodiment the latch component 406 (FIGS. 5B and 5C) independently engages (i) the shaft 666 and the shaft end portion 668 (via, e.g., a shaft notch 645b of the shaft end portion 668, shown in FIGS. 6B and 6C), and (ii) the elongate body 658 and the handle 654 (via, e.g., a handle notch 645a of the handle 654, shown in FIGS. 6B and 6C), such that the latch component 406 can (i) hold the shaft 666 and the shaft end portion 668 stationary and (ii) allow the elongate body 658 and the handle 654 to move in the second direction D2. This operation is described in greater detail below regarding FIGS. 6B and 6C. The interaction (e.g., force, friction, interference fit, or the like) between the coupling portion 659 of the implantable device 30 and the coupling elements 660 of the elongate body 658 can at least partially prevent the elongate body 658/handle 654 from moving in the second direction D2 unless or until a force sufficient to overcome this interaction is applied to the elongate body 658/handle 654. When the coupling portion 659 of the implantable device 30 is positioned within the chamber 657, moving the handle 645/coupling elements 660 in the second direction D2 can drive the coupling portion 659 into contact with/against the shaft 666. Continued movement of the coupling elements 660 in the second direction D2 can displace the implantable device 30 relative to one or more of the coupling elements 660 to release the implantable device 30 from the implantable delivery assembly 650. Because the shaft 666 can contact the coupling portion 659 of the implantable device 30 during the decoupling process, the shaft 666 can maintain the position of the implantable device 30 such that the implantable device 30 is expected to be decouplable from the implantable delivery assembly 650 without or substantially without changing the position of the implantable device 30 (e.g., driving the implantable device 30 away from the assembly 650) relative to one or more of the patient's tissues (e.g., a target location proximate the patient's medial branch MB). As described in greater detail below, this approach is expected to improve the accuracy and/or consistency with which the implantable device 30 can be positioned at or near a target location within a patient.

FIG. 6B is an exploded perspective view of the handle 654 and the shaft end portion 668 of FIG. 6A, each configured in accordance with embodiments of the present technology. Additionally, although not shown in FIG. 6B, the shaft end portion 668 is coupled to the shaft 666 and the handle 654 is coupled to the elongate body 658, as described above with reference to FIG. 6A. When the handle 654 slidably receives the shaft end portion 668, the handle slot 645a can be at least partially aligned with the shaft slot 645b, as shown and described in greater detail below with reference to FIG. 6C.

FIG. 6C is a perspective of the handle 654 and the shaft end portion 668 of FIG. 6B, and selected elements of the delivery system 400 of FIG. 4A, in accordance with embodiments of the present technology. The handle slot 645a can include a handle slot end surface 647a that defines a distal terminus of the handle slot 645a. The shaft slot 645b can include a shaft slot end surface 647b that defines a distal terminus of the shaft slot 645b. In the illustrated embodiment, the length of the handle slot 645a is greater than the length of the shaft slot 645b, such that, when the handle 654 slidably receives the shaft end portion 668, the handle slot end surface 647a is positioned distally from the shaft slot end surface 647b. When the handle 654 and the shaft end portion 668 are positioned within the delivery system 400, the release component 406 (e.g., retaining surface 407, shown in FIG. 4B) independently engages the handle slot 645a and the shaft slot 645b. More specifically, the release component 406 can contact the shaft slot end surface 647b and hold the shaft end portion 668 stationary relative to the body 402 (i.e., unless or until the release component 406 is actuated, as described above with reference to FIGS. 4A, 4B and 5C). Additionally, because the handle slot end surface 647a is positioned distally from the shaft slot end surface 647b, the handle slot end surface 647a is also positioned distally from the release component 406 such that the handle 654 can be moved in the second direction D2 (e.g., proximally and/or toward the release component 406) until the handle slot end surface 647a is aligned with the shaft slot end surface 647b (e.g., when the handle slot end surface 647a contacts the release component 406). The motion of the handle 654 in the second direction D2 and relative to the shaft end portion 668 can release the implantable device 30 from the implantable delivery assembly 650, as described above with reference to FIG. 6A.

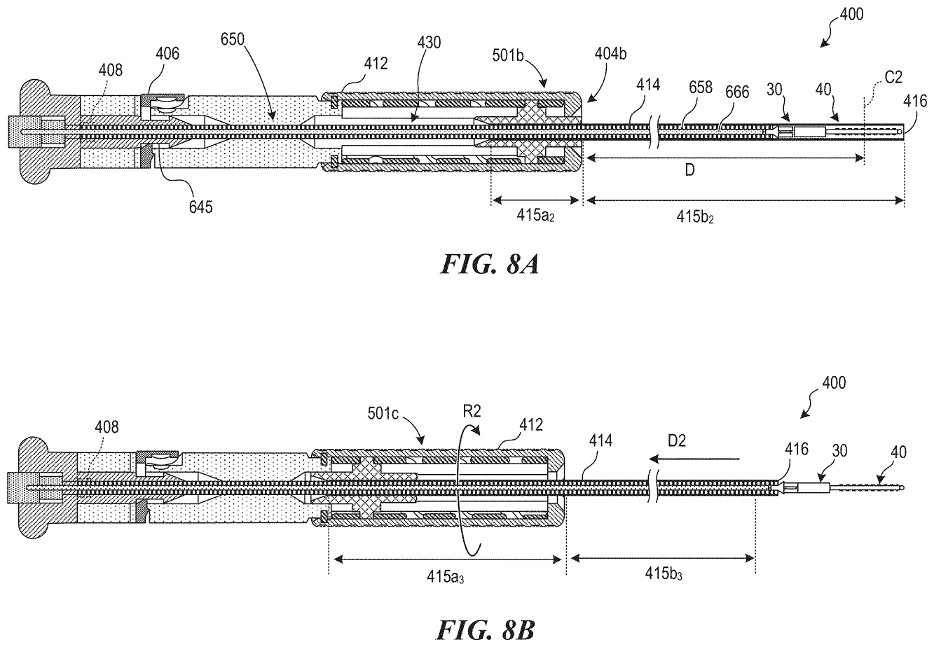

FIG. 7 is a side view of another implantable device insertion or delivery assembly 750 (“the implantable delivery assembly 750”) configured in accordance with embodiments of the present technology. FIG. 7 also includes an enlarged, cross-sectional view of the circled portion of the implantable delivery assembly 750 (shown in dashed lines). The implantable delivery assembly 750 can be generally similar to the implantable delivery assembly 650 of FIG. 6A, with like numbers (e.g., elongate body 758 versus the elongate body 658 of FIG. 6A) indicating like elements. However, the coupling component 756 of the implantable delivery assembly 750 is configured to receive a threaded coupling region 759 of the implantable device 30. In the illustrated embodiment, the threaded coupling region 759 can include internal threads 764, and the shaft 766 includes an externally threaded end portion 762 that corresponds to the threaded coupling region 759 and is positioned within chamber 757. The chamber 757 can be configured to at least partially prevent the implantable device 30 from rotating relative to the implantable delivery assembly 750. In the illustrated embodiment, for example, the chamber 757 includes a keyed surface 755 configured to receive the threaded coupling region 759. The shaft 766 can be coupled to a shaft actuator 768 operable to rotate the shaft 766 relative to the elongate body 758, as indicated by arrows R1 and R2. In the illustrated embodiment, the shaft actuator 768 is positioned at or proximate to the handle 754. In other embodiments, the shaft actuator 768 can have any other suitable position.

To couple the implantable device 30 and the implantable delivery assembly 750, the threaded coupling region 759 of the implantable device 30 can be at least partially inserted into the chamber 757, for example, until the threaded end portion 762 of the shaft 766 at least partially contacts the threaded coupling region 759. Next, the shaft actuator 768 can be rotated in the first direction R1 such that the threaded end portion 762 is threadably received by the corresponding internal threads 764 of the threaded coupling region 759. In the illustrated embodiment, the threaded end portion 762 is configured to be inserted into the threaded coupling region 759 of the implantable device 30. In other embodiments, the threaded coupling region 759 can be configured to be inserted into the threaded end portion of the shaft 766, or the threaded coupling region 759 and/or threaded end portion 762 can have any other suitable configuration.

To uncouple the implantable device 30, one or more steps of the coupling process can be performed in reverse. For example, the shaft actuator 768 can be rotated in a direction opposite the first direction R1 (e.g., the second direction R2) to unscrew the threaded end portion 762 from the threaded coupling region 759. Once the threaded end portion 762 is removed from the threaded coupling region 759, the implantable device can be removed from the chamber 757. Because the shaft 766 is rotated relative to the implantable device 30 during the decoupling process, the implantable device 30 is expected to be stationary or generally stationary during the decoupling process such that the implantable device 30 is expected to be decouplable from the implantable delivery assembly 750 without or substantially without changing the position of the implantable device 30 relative to one or more of the patient's tissues (e.g., a target location proximate the patient's medial branch MB). As described in greater detail below, this is expected to improve the accuracy and/or consistency with which the implantable device 30 can be position at or near a target location within a patient.

FIGS. 8A-8C are side cross-section views of the implantable delivery assembly 650 of FIG. 6A and the implantable device delivery system 400 of FIG. 4A, arranged in accordance with embodiments of the present technology. Although in the illustrated embodiments FIGS. 8A-8C include, and are described with reference to, the implantable delivery assembly 650 of FIG. 6A, the following description can apply, at least generally, to the implantable delivery assembly 750 of FIG. 7.

Referring to FIG. 8A, the implantable delivery assembly 650 can be inserted at least partially within the delivery system 400. While being inserted, the notch 645 can operably engage the latch component 406 to secure the implantable delivery assembly 650 relative to the delivery system 400. In at least some embodiments, the implantable delivery assembly 650 can be inserted after the navigation assembly 540 (FIGS. 5A-5C) has been removed from the delivery system 400 and/or while the delivery system 400 is in the second configuration 501b (initially described above with reference to FIG. 5B). In the second configuration 501b, the implantable device 30 can be positioned fully within the adjustable cannula 414, as shown in FIG. 8A. In the illustrated embodiment, the implantable device 30 is advanced at least partially though the overall lumen 430 using the implantable delivery assembly 650. In other embodiments, the implantable device 30 can be advanced at least partially through the delivery system 400 via a hydraulic pressure applied from the proximal end of the delivery system 400. Once inserted within the delivery system 400, the center C2 of the electrode array is located at the distance D from the second end portion 404b of the housing 402, e.g., the same distance as the center C1 of the navigation electrode 549 shown in FIG. 5B.

Referring to FIG. 8B, the delivery system 400 can be transitioned to a third configuration 501c to expose the implantable device 30, including the signal delivery device 40, such that the signal delivery device 40 can stimulate patient tissues to induce a patient response. Accordingly, in the third configuration 501c, the signal delivery device 40 can be positioned distally from the distal end terminus 416 of the adjustable cannula 414. In the illustrated embodiment, transitioning the delivery system 400 to the third configuration 501c includes rotating the actuator 412 in the second direction R2 to retract the distal end terminus 416 in the second direction D2, as described previously regarding, e.g., FIG. 4B. Transitioning the delivery system 400 from the second configuration 501b (FIG. 8A) to the third configuration 501c decreases the outer length 415b of the adjustable cannula 414 from the second outer length 415b₂ (FIG. 8A) to a third outer length 415b3, and correspondingly increases the inner length 415a from the second inner length 415a₂ (FIG. 8A) to a third inner length 415a3. The third outer length 415b3 is accordingly less than the first outer length 415b₁ (FIG. 5B), and the third inner length 415a3 is accordingly greater than the first inner length 415ai.

Referring to FIG. 8C, to decouple the implantable device 30 from the implantable delivery assembly 650, the handle 654 can be moved in the second direction D2 such that the coupling portion 659 of the implantable device 30 is driven against shaft 666 while the shaft 666 is held stationary, e.g., to release the implantable device 30 from the implantable delivery assembly 650 as described previously regarding FIGS. 6A-6C.

Accordingly, as described above with reference to FIGS. 4A-8C, the delivery system 400 is expected to position the implantable device 30 at the target location with improved accuracy, improved repeatability, and/or reduced migration of the implantable device 30. In some aspects of the present technology, and as described above, the center C2 of the implantable device's electrode array can be located at the same distance D from the second end portion 404b of the housing 402 as the center C1 of the navigation electrode 549. Accordingly, a practitioner can use the navigation electrode 549 to identify the target location based on the patient's response, replace the navigation electrode 549 with the implantable device 30, and expect the implantable device's electrode array to be centered at or near the target location and elicit a generally similar or a same response in the patient. This arrangement can improve the practitioner's ability to accurately position/align the implantable device 30 relative to the target location so as to address the patient's condition. Additionally, as described above, one or both of the implantable device delivery assemblies 650, 750 can be configured to reduce/minimize changes to the position of the implantable device 30 during the decoupling process. This is expected to further reduce migration of the implantable device 30 and/or further improve the accuracy and/or repeatability with which the implantable device 30 can be positioned at or near the target location.

### 5. Procedure

FIG. 9 outlines an overall procedure 900 for implanting an implantable device, such as the implantable device 30 of the above Figures, using a dual-mode navigation approach that includes image-based navigation and stimulation-based navigation. This procedure can be used to deliver one or more implantable devices from either a single entry point, or multiple entry points. Although implanting a single implantable device is often sufficient to address the patient's condition, using multiple implantable devices can provide additional assurance that a suitable therapeutic location or locations will be identified and/or targeted. Several steps of the overall procedure are described with reference to FIGS. 1-8C. Additionally, several steps of the overall procedure are described with reference to the delivery system 400 of FIGS. 4A, 4B, 5B, 5C, and 8A-8C, the navigation assembly 540 of FIGS. 5A-5C, the implantable delivery assembly 650 of FIGS. 6A-6C and 8A-8C, and/or the implantable delivery assembly 750 of FIG. 7. It will be appreciated, however, that the overall procedure can be performed with other delivery assemblies, navigation assemblies, and/or implantable delivery assemblies. Blocks labeled in FIG. 9 correspond to one or more of the headings below.

### 5.1 Representative Materials

Basic surgical instruments (i.e., forceps, scalpels, etc.).
Ultrasound system, for example, with color Doppler capabilities, as a further example, a GE 12L ultrasound probe, and ultrasound gel.
Implantable device delivery system, such as the delivery system 400 of FIGS. 4A, 4B, 5A-5C, 8A-8C, or another suitable implantable device delivery system.
Navigation assembly, such as the navigation assembly 540 of FIG. 5A
Implantable device, such as the implantable device 30 of FIGS. 3B, 3C, 6, 7, and 8A-8C, or another suitable implantable device.
External power source wirelessly couplable to the implantable device.
Peripheral nerve stimulator couplable to the navigation assembly.
Implantable device delivery assembly, such as the implantable device delivery assembly 650 of FIG. 6A, the implantable device delivery assembly 750 of FIG. 7, or another suitable implantable device delivery assembly.

### 5.2 Preparation for the Procedure

Flush implantable device delivery system, navigation assembly, implantable device, and/or implantable device delivery assembly with sterile saline.
Connect implantable device 30 to implantable delivery assembly 650, 750.
Insert navigation assembly 540 into delivery system 400.

### 5.3 Preparation for the Patient

Place the patient in the supine position with head supported by, e.g., a foam ring, and the surgeon above the head of the bed. Ask patient to rotate head to the left or right, extending the neck comfortably.

### 5.4 Hypoglossal Nerve Localization and Identification of Relevant Anatomy (Block 902)

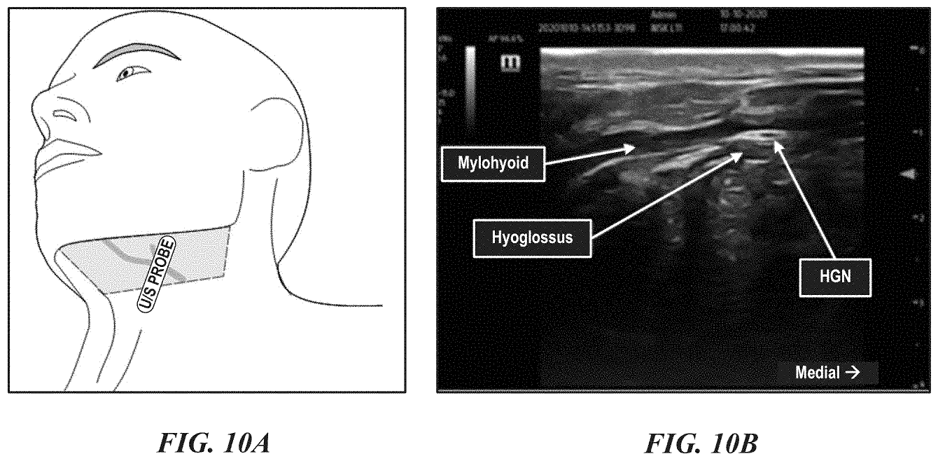

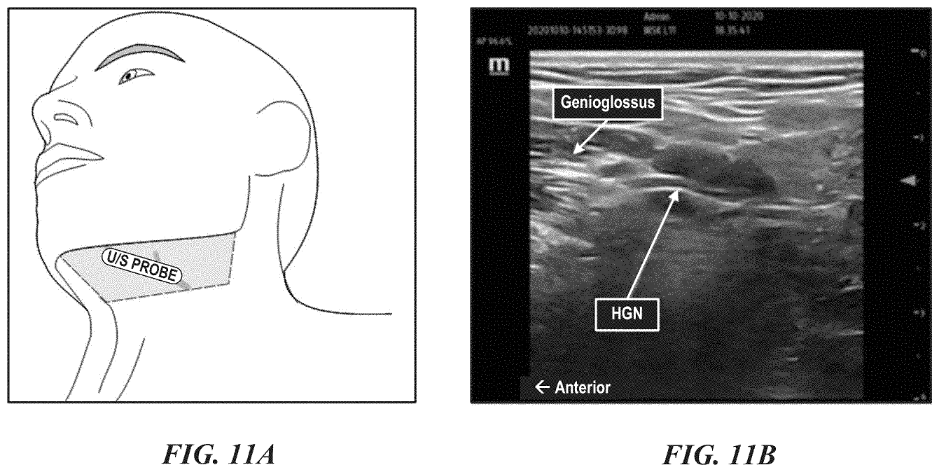

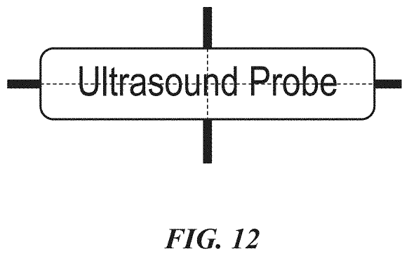

Place the ultrasound probe to lie between the hyoid bone and the approximate midpoint of the edge of the mandible, identify the hypoglossal nerve in the coronal view between the mylohyoid and hyoglossus muscles (FIGS. 10A, 10B).
While constantly maintaining the view of the HGN, rotate the probe to image a parasagittal view of the nerve with the longest visible length. Identify the leading edge of the hyoglossus muscle and the most distal portion of the nerve prior to diving into the genioglossus muscle (FIGS. 11A, 11B).
Using ultrasound, e.g., color Doppler ultrasound, identify vasculature in the area.
Identify submandibular and sublingual salivary glands in ultrasound imaging.
Identify the optimal insertion point that will allow for implantable device to be placed as close to parallel to the nerve as possible.
   External needle guide may be used to better align the insertion point to the ultrasound image.
   Investigate if pushing or pulling the submandibular or intraoral tissues would improve the parallel alignment between the implant tool/lead pathway and HGN. If such a configuration exists, apply the necessary tissue manipulation with available tools.
Using a skin marker, mark the position of the probe by marking the ends and the center of the probe (FIG. 12).

### 5.5 Administration of Anesthesia (Block 904)

Administer Conscious Sedation or General Anesthesia as indicated by the clinician and consented to by the patient.

### 5.6 Navigation Assembly Insertion (Block 906)

With the navigation assembly 540 inserted in the delivery system 400, transition the delivery system 400 to the first configuration 501a to expose the stimulating component 546.
Insert stimulating component 546 into patient.
Tip 547 of stimulating component 546 may be used to initially penetrate the patient's skin.

### 5.7 Image-Based Navigation (Block 908)

Using ultrasound to image the stimulating component 546, align the trajectory of the stimulating component 546 as close to the HGN as possible. The leading edge of the hyoglossus muscle and the most distal portion of the hypoglossal nerve that is visible may be used as the most proximal and distal references for the trajectory.
To achieve an angle as parallel as possible along the HGN, stimulating component may be inserted normal to the skin or at an exaggerated angle then tilted to the desired angle, as discussed above with reference to FIG. 3B. For example, the stimulating component 546 may be reoriented posteriorly to advance toward HGN through the Mylohyoid muscle plane.
   Mylohyoid muscle plane penetration can be visualized using ultrasound
   The Mylohyoid muscle tissue can deform or “tent” in response to contact with the tip 547 of the stimulating component 546.
   Once the Mylohyoid muscle is penetrated, the trajectory of the stimulation component 546 can be adjusted as needed based on Ultrasound imaging.
If inserting the stimulating component 546 posterior to the target area of the nerve, the insertion point should be aligned with the center plane of the ultrasound probe and 5-30 mm posterior to the posterior end of the ultrasound probe.
If inserting the stimulating component 546 anterior to the target area of the nerve, the insertion point should be aligned with the center plane of the ultrasound probe and posterior to the inner edge of the mandible.
Observe insertion procedure and check for excessive blood flow.
If needed, enlarge puncture hole slightly with stab incision along adjustable cannula 414 to sufficiently advance the stimulating component 546 without being hindered by the skin.

### 5.8 Stimulation-Based Navigation (Block 910)

Switch from primary navigation via Ultrasound imaging to primary navigation using stimulation pulses and patient response.
   The use of stimulation pulses to aid navigation can be referred to as “stim-nav”.
   Stim-nav can provide direct visible physical movement confirmation of the stimulation target (e.g., nerve and/or muscle tissue) and permits fine adjustment of the stimulating component 546 trajectory toward a position in which applied stimulation elicits the desired patient response (e.g., motor response, nerve twitch, etc.).
   Stim-nav can provide confirmation of delivery precision and demonstrate intended clinical outcome of placement and/or facilitate positional adjustments of the stimulation component 546 if the desired patient response is not achieved.
   Ultrasound, or other image-based navigation, may be used to confirm the position of the stimulation component 546. However, the ultrasound image of the target location can be compromised by the presence of the stimulation component 546, which can at least partially obscure or “shadow” the patient's neuroanatomy.
More generally, applying stimulation prior to implanting the implantable device can be an important navigation method for identifying and/or confirming the right location to elicit the desired response, and therefore locate one or more potential locations for implantation of the implantable device 30.
Connect the stimulating component 546 to a peripheral nerve stimulator. Use a sterile cover if necessary.
Slowly increase stimulation amplitude, looking for protrusion of the tongue (e.g., genioglossus activation) and/or minimal retrusion or dipping of the oral tongue inferiorly (e.g., styloglossus and hyoglossus activation).
In some embodiments, the navigation electrode 549 of the stimulation component 546 can be configured to deliver electrical stimulation having one or more of the following parameters:
   Amplitude=0.25 to 5 volts, such as 1.5 volts
   Pulse widths=25 to 250 μs, such as 150 μs
   Frequency=1 to 50 Hz, such as 40 Hz, or 2-3 Hz.
Stimulation can be delivered at predetermined times and/or in response to a command by the practitioner.
If no response or undesired response is seen, turn off stimulation and adjust the stimulation component 546 position slightly, e.g., under ultrasound guidance.

### 5.9 Navigation Assembly Removal (Block 912)

Once an appropriate stimulation response is achieved, disconnect the stimulation component 546 from the peripheral nerve stimulator.
While holding the housing 402 of the delivery system 400 in place:
   Transition the delivery system 400 from the first configuration 501a to the second configuration 501b, advancing the adjustable cannula 414 to cover the stimulation component 546.
   Using the latch component 406, uncouple the navigation assembly 540 from the delivery system 400.
   Withdraw the navigation assembly 540 from the delivery system 400 while leaving the delivery system 400 in position.

### 5.10 Implantable Device Insertion (Block 914)

While holding the delivery system 400 in position, insert implantable device 30 through the delivery system 400 using the implantable delivery assembly 650, 750.
The delivery system 400 can be configured such that, when the implantable device 30 is inserted through the delivery system 400, the center C2 of the signal delivery device 40 has the same or approximately the same position as the center C1 of the navigation electrode 549 of the stimulation component 546. Accordingly, when inserted through the delivery system 400, the signal delivery device 40 can be centered at or proximate to the position identified using the navigation assembly in block 910. This can improve the consistency with which the implantable device 30 is positioned at or proximate the target implant position.
The implantable device 30 is inserted while the delivery system 400 remains in the second configuration 501b and, accordingly, the implantable device 30 can be positioned fully in the adjustable cannula 414 once insertion is complete.

### 5.11 Confirm Implantable Device Positioned at the Target Location (Block 916)

Holding the housing 402 of the delivery system 400 in place, transition the delivery system 400 from the second configuration 501b to the third configuration 501c until the adjustable cannula 414 is retracted to expose the implantable device 30.
Confirm that the electrode array has not migrated.
   If the shadow cast by the electrode array doesn't obscure the HGN, then use ultrasound to confirm the position of the electrode array relative to the HGN.
   Optionally, deliver electrical stimulation to the patient via the implantable device 30 to confirm the implantable device's position, as described previously herein and below.
Connect the implantable device to the external power source.
Provide stimulus to the patient using the implantable device 30 to confirm that the implantable device is positioned at the target location. This process can be generally similar to or the same as “Stimulation-Based Navigation” block 910.
   In some embodiments, the adjustable cannula 414 can be made of one or more RF permeable materials and or include one or more through slots or windows to allow electrical energy conduction to tissue from the signal delivery device 40 while the delivery system 400 is in the second configuration 501b. It may be difficult to reposition the signal delivery device 40 when the delivery system 400 is in the third configuration 501c (e.g., when the signal delivery device 40 is exposed). Accordingly, an RF-permeable cannula can allow the signal delivery device 40 to be more easily repositioned while also delivering electrical stimulation to the patient.
In some embodiments, the implantable device 30 can be put into a “stim nav” mode, where a first/leading electrode is negative, a second/trailing electrode is positive, and a stimulation signal is applied between two electrodes (e.g., the leading electrode and the trailing electrode. Stimulation parameters include a frequency of at 1 Hz-3 Hz, a current of 2 mA-5 mA, and a pulse width of 100 μs-250 μs.
If the implantable device 30 includes a signal delivery device 40 having one or more directional electrodes, such as described with reference to FIGS. 3C and 3D of U.S. patent application Ser. No. 17/749,025, incorporated by reference herein, the orientation of the directionally biased electrodes relative to the medial branch MB can be optimized by turning implantable device 30 via the delivery system 400 and/or the implantable delivery assembly 650, 750.
If the desired patient response is not observed, the implantable delivery assembly can be removed from the delivery system 400, the navigation assembly 540 can be reinserted into the delivery system 400, and the target location can be identified, e.g., as described previously regarding block 910.

### 5.12 Implant the Implantable Device (Block 918)

Once confirmed, release the implantable device 30 from the implantable delivery assembly as described previously regarding FIGS. 6A-6C and 7.
Anchor the implantable device.
Remove the implantable delivery assembly from the delivery system 400.
Remove the delivery system 400 from the patient.
Prepare the wound site or suture.
Begin post procedure patient recovery.

The procedure described above is expected to provide several advantages. For example, the implantable device 30 can be positioned along and adjacent to the path of muscle contraction upon stimulation (or swallowing or breathing). This can reduce patient discomfort after implantation and/or reduce migration of the implantable device 30. Additionally, implanting the implantable device as described above can position the implantable device's power receiving antenna closer to the skin surface and superior to the neural target. When multiple implantable devices are implanted, this technique can allow a single and/or smaller area power source antenna to be placed over and provide power to or more implantable devices.

From the foregoing, it will be appreciated that specific embodiments of the disclosed technology have been described herein for purposes of illustration, but that various modifications may be made without deviating from the technology. Other representative targets for the stimulation include palatoglossal stimulation, cranial nerve stimulation, phrenic nerve stimulation, direct palatoglossus muscle stimulation, hyolaryngeal stimulation, and/or glossopharyngeal nerve stimulation. Certain aspects of the technology described in the context of particular embodiments may be combined or eliminated in other embodiments. Further, while advantages associated with certain embodiments of the disclosed technology have been described in the context of those embodiments, other embodiments may also exhibit such advantages, and not all embodiments need necessarily exhibit such advantages to fall within the scope of the technology. Accordingly, the disclosure and associated technology can encompass other embodiments not expressly shown or described herein.

As used herein, the phrase “and/or,” as in “A” and/or “B” refers to A alone, B alone and both A and B. To the extent any materials incorporated herein by reference conflict with the present disclosure, the present disclosure controls.

The following examples provide additional representative features of the present technology.

### Examples

1. A delivery system for positioning an implantable device at least proximate to a target location in a patient using dual-mode navigation, the delivery system comprising:
   a body having a first end portion and a second end portion opposite the first end portion, the body defining a first lumen that extends at least partially between the first end portion and the second end portion;
   an adjustable cannula defining a second lumen, the adjustable cannula including (i) a first portion positioned within the first lumen and (ii) a second portion extending beyond the first lumen, the second lumen being at least partially aligned with the first lumen to form an overall lumen of the delivery system;
   an actuation mechanism operably coupled to the body and the adjustable cannula, the actuation mechanism configured to increase or decrease a length of the second portion of the adjustable cannula, the actuation mechanism including—
      a driven element coupled to the first portion of the adjustable cannula and slidably disposed within the first lumen of the body, the driven element including a drive tab,
      a drive element rotatably coupled to the body and including at least one helical drive slot sized to slidably receive the drive tab, and
      an actuator coupled to the drive element and rotatably coupled to the body, the actuator configured to rotate about a longitudinal axis of the body to cause a corresponding rotation of the drive element to slidably translate the driven element along the longitudinal axis to increase or decrease the length of the second portion of the adjustable cannula,
      wherein the actuator is configured to move the adjustable cannula between—
         a first configuration in which the second portion has a first length,
         a second configuration in which the second portion has a second length greater than the first length, and
         a third configuration in which the second portion has a third length less than the first length;
   a navigation assembly including a first elongate body having a proximal handle and an electrically activatable distal stimulating tip, the navigation assembly sized to be at least partially insertable into the overall lumen of the delivery system; and
   an implantable device delivery assembly including a second elongate body having a proximal actuating handle and a distal implantable device coupling feature, the distal implantable device coupling feature releasably couplable to the implantable device, the implantable device delivery assembly sized to be at least partially insertable into the overall lumen of the delivery system;
   wherein—
      in the first configuration, the distal stimulating tip extends fully beyond the adjustable cannula when the navigation assembly is inserted into the overall lumen and the implantable device also extends fully beyond the adjustable cannula when the implantable device delivery assembly is inserted into the overall lumen;
      in the second configuration, the distal stimulating tip extends fully beyond the adjustable cannula when the navigation assembly is inserted into the overall lumen and the implantable device is positioned partially within the adjustable cannula when the implantable device delivery assembly is inserted into the overall lumen; and
      in the third configuration, the distal stimulating tip is positioned fully within the adjustable cannula when the navigation assembly is inserted into the overall lumen and the implantable device is also positioned fully within the adjustable cannula when the implantable device delivery assembly is inserted into the overall lumen.
2. The delivery system of example 1 wherein—
the distal stimulating tip of the navigation assembly includes at least one electrode;
the implantable device includes an electrode array;
when the navigation assembly is inserted into the overall lumen, the at least one electrode is positioned at a distance from the second end portion of the body; and
when the implantable device delivery assembly is inserted into the overall lumen, a center of the electrode array is at the distance from the second end portion of the body.
3. The delivery system of example 1 or example 2 wherein rotating the actuator in a first rotary direction moves the adjustable cannula from the first configuration toward the second configuration, and wherein rotating the actuator in a second rotary direction opposite the first rotary direction moves the adjustable cannula from the second configuration toward the third configuration.
4. A delivery system, comprising:
a body having an at least partially hollow interior;
an adjustable sheath defining a lumen, the adjustable sheath including (i) a first portion positioned within the interior of the body and (ii) a second portion extending beyond the interior; and
an actuation mechanism operably coupled to the body and the adjustable sheath, the actuation mechanism being operable to move the adjustable sheath between—a first configuration in which the second portion has a first length,
   a second configuration in which the second portion has a second length greater than the first length, and
   a third configuration in which the second portion has a third length less than the first length.
5. The delivery system of example 4, further comprising a navigation assembly including an electrically active stimulating tip, the navigation assembly configured to be at least partially insertable into the lumen via the interior of the body.
6. The delivery system of example 5, wherein the body includes a latch component configured to releasably couple to the navigation assembly to at least partially prevent movement of the navigation assembly relative to the delivery system.
7. The delivery system of example 6 wherein the latch component is biased in a first radial direction relative to a longitudinal axis of the body, and wherein moving the latch component in a second radial direction opposite the first radial direction uncouples the navigation assembly and the latch component.
8. The delivery system of example 5 or example 6 wherein, in the first configuration, the electrically active stimulating tip extends beyond an end terminus of the adjustable cannula, and wherein, in the second configuration, the electrically active stimulating tip is positioned within the adjustable cannula.
9. The delivery system of any of examples 4-8, further comprising an implantable device delivery assembly including a coupling feature configured to be releasably couplable to an implantable device, the implantable device delivery assembly configured to be at least partially insertable into the lumen via the interior of the body.
10. The delivery system of example 9, further comprising the implantable device, and wherein the implantable device is a leadless device.
11. The delivery system of example 9 or example 10 wherein, in the first configuration, the implantable device delivery assembly is configured to position the implantable device within the adjustable cannula, and wherein, in the third configuration, the implantable device delivery assembly is configured to position an electrode array of the implantable device to extend beyond an end terminus of the adjustable cannula.
12. The delivery system of any of examples 9-11 wherein the coupling feature includes a threaded coupling feature configured to be threadably received by the implantable device.
13. The delivery system of any of examples 9-12 wherein the coupling feature includes a plurality of recesses positioned around a chamber, wherein the implantable device includes a plurality of tabs, wherein individual ones of the plurality of recesses are configured to receive a corresponding one of the plurality of tabs when at least part of the implantable device is positioned within the chamber.
14. A method of addressing a patient's sleep apnea by positioning an implantable device at least proximate to a target location using dual-mode navigation, the method comprising:
inserting a navigation assembly into a delivery system with an adjustable sheath of the delivery system in a first configuration such that a stimulating tip of the navigation assembly extends beyond the adjustable sheath;
using image-based navigation to move the stimulating tip toward a first position within the patient, wherein moving the stimulating tip toward the first position includes penetrating the patient's mylohyoid muscle;
using stimulation-based navigation to move the stimulating tip toward a second position at least proximate to the target location, wherein using the stimulation-based navigation includes—
   delivering one or more electrical navigation signals to the patient via the stimulating tip,
   observing a response of the patient to individual ones of the one or more electrical navigation signals, and
   based on the response, repositioning the stimulating tip and/or adjusting an amplitude of individual ones of the electrical navigation signals;
rotating an actuator of the delivery system in a first direction to extend the adjustable sheath of the delivery system from the first configuration to a second configuration in which the stimulating tip is positioned within the adjustable sheath;
with the adjustable sheath in the second configuration—
   removing the navigation assembly from the delivery system, and
   inserting an implantable device delivery assembly into the delivery system, the implantable device delivery assembly including the implantable device, the implantable device positioned within the adjustable sheath;
rotating the actuator in a second direction opposite the first direction to retract the adjustable sheath from the second configuration to a third configuration in which the implantable device extends beyond the adjustable sheath;
delivering an electrical signal to the patient via the implantable device to determine whether the implantable device is positioned at least proximate to the target location; and
when the implantable device is determined to be positioned proximate the target location, actuating a release mechanism of the implantable device delivery assembly to release the implantable device from the delivery system.
15. The method of example 14 wherein one or both of using image-based navigation to move the stimulating tip and using stimulation-based navigation to move the stimulating tip includes moving the stimulating tip in an anterior-to-posterior direction.
16. The method of example 14 wherein one or both of using image-based navigation to move the stimulating tip and using stimulation-based navigation to move the stimulating tip includes moving the stimulating tip in a posterior-to-anterior direction.
17. The method of any of examples 14-16 wherein—
using image-based navigation to move the stimulating tip including using image-based navigation to move the stimulating tip along a first trajectory and toward the first position, and
using stimulation-based navigation to move the stimulating tip includes using stimulation-based navigation to move the stimulating tip along a second trajectory different than the first trajectory and toward the second position.
18. The method of example 17, further comprising repositioning the delivery system to reorient the stimulating tip from the first trajectory toward the second trajectory.
19. The method of any of examples 14-18 wherein the target location includes a genioglossus muscle of the patient.
20. The method of any of examples 14-18 wherein the target location includes a hypoglossal nerve of the patient.
21. The method of any of examples 14-18 wherein the target location includes an ansa cervicalis nerve of the patient.
22. A method of addressing a patient's sleep apnea, the method comprising:
using image-based navigation to move a stimulating tip of a delivery system toward a first position within the patient;
using stimulation-based navigation to move the stimulating tip toward a second position at least proximate a target location within the patient;
moving an adjustable sheath of the delivery system from a first configuration in which the stimulating tip extends beyond the adjustable sheath to a second configuration in which the stimulating tip is positioned within the adjustable sheath;
with the adjustable sheath in the second configuration—
   removing the stimulating tip from the delivery system, and
   inserting an implantable device into the delivery system, the implantable device positioned within the adjustable sheath;
moving the adjustable sheath from the second configuration to a third configuration in which the implantable device extends beyond the adjustable sheath;
delivering an electrical signal to the patient via the implantable device to determine whether the implantable device is positioned at least proximate to the target location; and
when the implantable device is determined to be positioned at least proximate to the target nerve, releasing the implantable device from the delivery system.
23. The method of example 22, further comprising, prior to using the image-based navigation to move the stimulating tip, inserting the stimulating tip through the adjustable sheath to position an electrode of the stimulating tip at a distance beyond a body of the delivery system, wherein inserting the implantable device into the delivery system includes positioning a center of an electrode array of the delivery system at the distance beyond the body of the delivery system.
24. The method of example 22 or example 23 wherein releasing the implantable device includes moving a handle portion of an implantable device delivery assembly relative to a shaft of the implantable device delivery assembly to uncouple the implantable device from the implantable device delivery assembly.
25. The method of any of examples 22-24 wherein using image-based navigation includes using an ultrasound probe to visualize a position of the stimulating tip, and wherein using stimulation-based navigation includes delivering, via the stimulating tip, one or more electrical signals to the patient's tissue.
26. The method of any of examples 22-25 wherein the target location includes a hypoglossal nerve of the patient.
27. The method of any of examples 22-25 wherein the target location includes an ansa cervicalis nerve of the patient.
28. The method of any of examples 22-25 wherein the target location includes a genioglossus muscle of the patient.

I/We claim:

1. A delivery system for positioning an implantable device at least proximate to a target location in a patient using dual-mode navigation, the delivery system comprising:
a body having a first end portion and a second end portion opposite the first end portion, the body defining a first lumen that extends at least partially between the first end portion and the second end portion;
an adjustable cannula defining a second lumen, the adjustable cannula including (i) a first portion positioned within the first lumen and (ii) a second portion extending beyond the first lumen, the second lumen being at least partially aligned with the first lumen to form an overall lumen of the delivery system;
an actuation mechanism operably coupled to the body and the adjustable cannula, the actuation mechanism configured to increase or decrease a length of the second portion of the adjustable cannula, the actuation mechanism including—
a driven element coupled to the first portion of the adjustable cannula and slidably disposed within the first lumen of the body, the driven element including a drive tab,
a drive element rotatably coupled to the body and including at least one helical drive slot sized to slidably receive the drive tab, and
an actuator coupled to the drive element and rotatably coupled to the body, the actuator configured to rotate about a longitudinal axis of the body to cause a corresponding rotation of the drive element to slidably translate the driven element along the longitudinal axis to increase or decrease the length of the second portion of the adjustable cannula,
wherein the actuator is configured to move the adjustable cannula between—a first configuration in which the second portion has a first length,
a second configuration in which the second portion has a second length greater than the first length, and
a third configuration in which the second portion has a third length less than the first length;
a navigation assembly including a first elongate body having a proximal handle and an electrically activatable distal stimulating tip, the navigation assembly sized to be at least partially insertable into the overall lumen of the delivery system; and
an implantable device delivery assembly including a second elongate body having a proximal actuating handle and a distal implantable device coupling feature, the distal implantable device coupling feature releasably couplable to the implantable device, the implantable device delivery assembly sized to be at least partially insertable into the overall lumen of the delivery system;
wherein—
in the first configuration, the distal stimulating tip extends fully beyond the adjustable cannula when the navigation assembly is inserted into the overall lumen and the implantable device also extends fully beyond the adjustable cannula when the implantable device delivery assembly is inserted into the overall lumen;
in the second configuration, the distal stimulating tip extends fully beyond the adjustable cannula when the navigation assembly is inserted into the overall lumen and the implantable device is positioned partially within the adjustable cannula when the implantable device delivery assembly is inserted into the overall lumen; and
in the third configuration, the distal stimulating tip is positioned fully within the adjustable cannula when the navigation assembly is inserted into the overall lumen and the implantable device is also positioned fully within the adjustable cannula when the implantable device delivery assembly is inserted into the overall lumen.

2. The delivery system of claim 1 wherein—
the distal stimulating tip of the navigation assembly includes at least one electrode;
the implantable device includes an electrode array;
when the navigation assembly is inserted into the overall lumen, the at least one electrode is positioned at a distance from the second end portion of the body; and
when the implantable device delivery assembly is inserted into the overall lumen, a center of the electrode array is at the distance from the second end portion of the body.

3. The delivery system of claim 1 wherein rotating the actuator in a first rotary direction moves the adjustable cannula from the first configuration toward the second configuration, and wherein rotating the actuator in a second rotary direction opposite the first rotary direction moves the adjustable cannula from the second configuration toward the third configuration.

4. A delivery system, comprising:
a body having an at least partially hollow interior;
an adjustable sheath defining a lumen, the adjustable sheath including (i) a first portion positioned within the interior of the body and (ii) a second portion extending beyond the interior; and
an actuation mechanism operably coupled to the body and the adjustable sheath, the actuation mechanism being operable to move the adjustable sheath between—a first configuration in which the second portion has a first length,
a second configuration in which the second portion has a second length greater than the first length, and
a third configuration in which the second portion has a third length less than the first length.

5. The delivery system of claim 4, further comprising a navigation assembly including an electrically active stimulating tip, the navigation assembly configured to be at least partially insertable into the lumen via the interior of the body.

6. The delivery system of claim 5, wherein the body includes a latch component configured to releasably couple to the navigation assembly to at least partially prevent movement of the navigation assembly relative to the delivery system.

7. The delivery system of claim 6 wherein the latch component is biased in a first radial direction relative to a longitudinal axis of the body, and wherein moving the latch component in a second radial direction opposite the first radial direction uncouples the navigation assembly and the latch component.

8. The delivery system of claim 5 wherein, in the first configuration, the electrically active stimulating tip extends beyond an end terminus of the adjustable cannula, and wherein, in the second configuration, the electrically active stimulating tip is positioned within the adjustable cannula.

9. The delivery system of claim 4, further comprising an implantable device delivery assembly including a coupling feature configured to be releasably couplable to an implantable device, the implantable device delivery assembly configured to be at least partially insertable into the lumen via the interior of the body.

10. The delivery system of claim 9, further comprising the implantable device, and wherein the implantable device is a leadless device.

11. The delivery system of claim 9, further comprising the implantable device, wherein, in the first configuration, the implantable device delivery assembly is configured to position the implantable device within the adjustable cannula, and wherein, in the third configuration, the implantable device delivery assembly is configured to position an electrode array of the implantable device to extend beyond an end terminus of the adjustable cannula.

12. The delivery system of claim 9 wherein the coupling feature includes a threaded coupling feature configured to be threadably received by the implantable device.

13. The delivery system of claim 9 wherein the coupling feature includes a plurality of recesses positioned around a chamber, wherein the implantable device includes a plurality of tabs, wherein individual ones of the plurality of recesses are configured to receive a corresponding one of the plurality of tabs when at least part of the implantable device is positioned within the chamber.

14. A method of addressing a patient's sleep apnea by positioning an implantable device at least proximate to a target location using dual-mode navigation, the method comprising:
inserting a navigation assembly into a delivery system with an adjustable sheath of the delivery system in a first configuration such that a stimulating tip of the navigation assembly extends beyond the adjustable sheath;
using image-based navigation to move the stimulating tip toward a first position within the patient, wherein moving the stimulating tip toward the first position includes penetrating the patient's mylohyoid muscle;
using stimulation-based navigation to move the stimulating tip toward a second position at least proximate to the target location, wherein using the stimulation-based navigation includes—
delivering one or more electrical navigation signals to the patient via the stimulating tip,
observing a response of the patient to individual ones of the one or more electrical navigation signals, and
based on the response, repositioning the stimulating tip and/or adjusting an amplitude of individual ones of the electrical navigation signals;
rotating an actuator of the delivery system in a first direction to extend the adjustable sheath of the delivery system from the first configuration to a second configuration in which the stimulating tip is positioned within the adjustable sheath;
with the adjustable sheath in the second configuration—removing the navigation assembly from the delivery system, and
inserting an implantable device delivery assembly into the delivery system, the implantable device delivery assembly including the implantable device, the implantable device positioned within the adjustable sheath;
rotating the actuator in a second direction opposite the first direction to retract the adjustable sheath from the second configuration to a third configuration in which the implantable device extends beyond the adjustable sheath;
delivering an electrical signal to the patient via the implantable device to determine whether the implantable device is positioned at least proximate to the target location; and
when the implantable device is determined to be positioned proximate the target location, actuating a release mechanism of the implantable device delivery assembly to release the implantable device from the delivery system.

15. The method of claim 14 wherein one or both of using image-based navigation to move the stimulating tip and using stimulation-based navigation to move the stimulating tip includes moving the stimulating tip in an anterior-to-posterior direction.

16. The method of claim 14 wherein one or both of using image-based navigation to move the stimulating tip and using stimulation-based navigation to move the stimulating tip includes moving the stimulating tip in a posterior-to-anterior direction.

17. The method of claim 14 wherein—
using image-based navigation to move the stimulating tip including using image-based navigation to move the stimulating tip along a first trajectory and toward the first position, and
using stimulation-based navigation to move the stimulating tip includes using stimulation-based navigation to move the stimulating tip along a second trajectory different than the first trajectory and toward the second position.

18. The method of claim 17, further comprising repositioning the delivery system to reorient the stimulating tip from the first trajectory toward the second trajectory.

19. The method of claim 14 wherein the target location includes a genioglossus muscle of the patient.

20. The method of claim 14 wherein the target location includes a hypoglossal nerve of the patient.

21. The method of claim 14 wherein the target location includes an ansa cervicalis nerve of the patient.

22. A method of addressing a patient's sleep apnea, the method comprising:
using image-based navigation to move a stimulating tip of a delivery system toward a first position within the patient;
using stimulation-based navigation to move the stimulating tip toward a second position at least proximate a target location within the patient;
moving an adjustable sheath of the delivery system from a first configuration in which the stimulating tip extends beyond the adjustable sheath to a second configuration in which the stimulating tip is positioned within the adjustable sheath;
with the adjustable sheath in the second configuration—
removing the stimulating tip from the delivery system, and
inserting an implantable device into the delivery system, the implantable device positioned within the adjustable sheath;
moving the adjustable sheath from the second configuration to a third configuration in which the implantable device extends beyond the adjustable sheath;
delivering an electrical signal to the patient via the implantable device to determine whether the implantable device is positioned at least proximate to the target location; and
when the implantable device is determined to be positioned at least proximate to the target nerve, releasing the implantable device from the delivery system.

23. The method of claim 22, further comprising, prior to using the image-based navigation to move the stimulating tip, inserting the stimulating tip through the adjustable sheath to position an electrode of the stimulating tip at a distance beyond a body of the delivery system, wherein inserting the implantable device into the delivery system includes positioning a center of an electrode array of the delivery system at the distance beyond the body of the delivery system.

24. The method of claim 22 wherein releasing the implantable device includes moving a handle portion of an implantable device delivery assembly relative to a shaft of the implantable device delivery assembly to uncouple the implantable device from the implantable device delivery assembly.

25. The method of claim 22 wherein using image-based navigation includes using an ultrasound probe to visualize a position of the stimulating tip, and wherein using stimulation-based navigation includes delivering, via the stimulating tip, one or more electrical signals to the patient's tissue.

26. The method of claim 22 wherein the target location includes a hypoglossal nerve of the patient.

27. The method of claim 22 wherein the target location includes an ansa cervicalis nerve of the patient.

28. The method of claim 22 wherein the target location includes a genioglossus muscle of the patient.
